(19) United States
(12) Patent Application Publication
(10) Pub. No.: US 2024/0268993 A1
(43) Pub. Date: Aug. 15, 2024

(54) MOUTHPIECE FOR TREATING MEDICAL CONDITION(S) AND/OR SLEEP MONITORING

(71) Applicant: Zerene Inc., Walnut Creek, CA (US)

(72) Inventor: Arash Abdollahi Sabet, Walnut Creek, CA (US)

(73) Assignee: Zerene Inc., Walnut Creek, CA (US)

(21) Appl. No.: 18/438,314

(22) Filed: Feb. 9, 2024

Related U.S. Application Data
(60) Provisional application No. 63/444,918, filed on Feb. 11, 2023.
(60) Provisional application No. 63/529,750, filed on Jul. 30, 2023.

(51) Int. Cl. A61F 5/56; A61B 5/00; A61B 5/145

(52) U.S. Cl. CPC A61F 5/566; A61B 5/14542; A61B 5/4818; A61B 5/682; A61B 2562/0219; A61B 2562/0271

(57) ABSTRACT

A mouthpiece includes: a body comprising one or more portions defining a space for accommodating one or more teeth of a user; and a first cavity at least partly in the body, wherein a volume inside the first cavity is variable in response to a compression applied to the body.

### RELATED APPLICATION DATA

This application claims priority to, and the benefit of, U.S. Provisional Patent Application No. 63/444,918, filed on Feb. 11, 2023, pending, and U.S. Provisional Patent Application No. 63/529,750, filed on Jul. 30, 2023, pending. The entire disclosures of the above applications are expressly incorporated by reference herein.

### FIELD

The field relates to apparatuses and methods for treating medical conditions, and more specifically to apparatuses and methods for addressing bruxing, snoring, sleep apnea, or other medical conditions, using a mouthpiece.

### BACKGROUND

Prevalence of sleep bruxism—excessive grinding or jaw clenching—is reported to be 9.7% to 15.9% in the general population. Symptoms include jaw muscle pain, headache, hypersensitive teeth, tooth wear, and damage to dental restorations. In children, the overall prevalence is 14-20%.

Most people who brux are unaware of the problem, either because there are no symptoms, or because the symptoms are not understood to be associated with a clenching and grinding problem. Bruxism may cause a variety of signs and symptoms, including: Excessive tooth wear, Tooth fractures, Hypersensitive teeth, Inflammation of the periodontal ligament of teeth; make them sore to bite on, Loosening of teeth, Grinding or tapping noise during sleep, Burning sensation on the tongue, Indentions of the teeth in the tongue, Hypertrophy of muscles of mastication, Generalized facial pain, Muscular fatigue or tenderness, Clicking of the temporomandibular joints, Headaches, Aching temples, and Temporomandibular joint wear.

Bruxism can cause significant tooth wear if it is severe, and sometimes dental restorations (crowns, fillings, etc.) are damaged or lost, sometimes repeatedly. Most dentists therefore prefer to keep dental treatment in people with bruxism very simple and only carry it out when essential, since any dental work is likely to fail in the long term. Dental implants, dental ceramics such as Emax crowns and complex bridgework for example are relatively contraindicated in bruxists. In the case of crowns, the strength of the restoration becomes more important, sometimes at the cost of aesthetic considerations. For example, a full coverage gold crown, which has a degree of flexibility and also involves less removal (and therefore less weakening) of the underlying natural tooth, may be more appropriate than other types of crowns which are primarily designed for esthetics rather than durability. Porcelain veneers on the incisors are particularly vulnerable to damage, and sometimes a crown can be perforated by occlusal wear.

Occlusal splints (dental guards) are commonly prescribed, mainly by dentists and dental specialists, as a treatment for bruxism. Proponents of their use claim many benefits, however when the evidence is critically examined in systematic reviews of the topic, it is reported that there is insufficient evidence to show that occlusal splints are effective for sleep bruxism. Furthermore, occlusal splints are probably ineffective for awake bruxism, since they tend to be worn only during sleep. However, occlusal splints may be of some benefit in reducing the tooth wear that may accompany bruxism, but by mechanically protecting the teeth rather than reducing the bruxing activity itself. In a minority of cases, sleep bruxism may be made worse by an occlusal splint. Some patients will periodically return with splints with holes worn through them, either because the bruxism is aggravated, or unaffected by the presence of the splint. When tooth-to-tooth contact is possible through the holes in a splint, it is offering no protection against tooth wear and needs to be replaced.

Bruxism is a pervasive, poorly controlled disease. The current standard of care is focused to minimize the effect of symptoms; instead of addressing the underlying disorder.

New apparatuses and methods for training users to address sleep bruxism, snoring, sleep apnea, and other disorders are described herein. An embodiment described herein utilizes biofeedback to implement a safe regimen for curing a medical condition.

### SUMMARY

An embodiment described herein is an apparatus having a mouthpiece (e.g., a splint), a processing module, and a tube (e.g., made from silicone) coupled between the mouthpiece and the processing module. The tube is positioned such that during teeth grinding (bruxing) the volume inside the tube or inside a pocket coupled to the tube decreases, resulting in increase of pressure that is detected by the processing module. The processing module is coupled to the end of the tube and is located outside the user's mouth during use. In some embodiments, the processing module is configured to detect bruxing activity and provide biofeedback, such as vibration, to the user's lips, teeth, gums, tongue, or any combination of the foregoing. Biofeedback allows the user to become aware (consciously, unconsciously, or semi-consciously) of a physiological activity, and to alter the physiological activity with the aim of improving health. Alternative or in addition to vibration, the apparatus may provide auditory and/or visual cues to alert (consciously, unconsciously, or semi-consciously) the user of the bruxing activity, and to train the user away from grinding his/her teeth.

In some embodiments, all electronics, batteries, processors, etc., of the apparatus are arranged outside the mouth of the user. This greatly reduces risks, relaxes design constraints around form factor and size, and dramatically reduces choking and biocompatibility risk. Even with the sensing electronics and feedback signal generator located outside the user's mouth, biofeedback can still be transmitted into the mouth of the user via a biocompatible link—such as the tube of the apparatus.

An apparatus includes: a mouthpiece configured for placement inside a mouth of a user; a processing module; and a tube having a first end coupled to the mouthpiece, and a second end coupled to the processing module; wherein the processing module comprises a sensor configured to detect a characteristic due to a biting force applied to the mouthpiece by the user.

Optionally, the mouthpiece comprises an inner wall, an outer wall, and a bite portion between the inner wall and the outer wall.

Optionally, the mouthpiece comprises a first cavity configured to be in fluid communication with a lumen of the tube.

Optionally, the first cavity is on one of a left side or a right side of the mouthpiece.

Optionally, the mouthpiece further comprises a spacer at another one of the left side or the right side of the mouthpiece.

Optionally, the mouthpiece further comprises a second cavity configured to be in fluid communication with the lumen of the tube.

Optionally, the tube has a Y-configuration.

Optionally, the first cavity is variable in response to a compression applied to the mouthpiece.

Optionally, the first cavity is between two or more layers of materials that are laminated together.

Optionally, the mouthpiece is a customized mouthpiece.

Optionally, the mouthpiece comprises at least two layers of materials laminated together.

Optionally, the tube is integrally formed with the mouthpiece.

Optionally, the first end of the tube is configured to detachably couple to the mouthpiece.

Optionally, the second end of the tube is configured to detachably couple to the processing module.

Optionally, the processing module comprises a locking mechanism configured to lock the second end of the tube with respect to the processing module.

Optionally, the tube is configured to maintain the processing module outside the mouth of the user while the mouthpiece is inside the mouth of the user.

Optionally, the tube is configured to provide the pressure to the processing module in response to the biting force.

Optionally, the tube has a rigidity that is sufficient for allowing the tube to transmit a vibrational force from the processing module to the user.

Optionally, the processing module comprises a feedback signal generator configured to provide feedback to the user.

Optionally, the feedback has an intensity sufficient to cause the user to stop or reduce the biting force.

Optionally, the intensity of the feedback is based on a failure or a success of a previous feedback to cause a reaction from the user.

Optionally, the processing module is configured to detect a snoring of the user, and wherein the feedback has an intensity sufficient to cause the user to stop the snoring.

Optionally, the feedback signal generator comprises a vibrational force generator configured to provide vibration energy as the feedback, and wherein the tube is configured to transmit the vibration energy to the user.

Optionally, the feedback signal generator comprises a sound generator configured to provide an audio output as the feedback.

Optionally, the feedback signal generator comprises a light generator configured to provide light as the feedback.

Optionally, the feedback signal generator comprises a current generator configured to provide an electric signal as the feedback.

Optionally, the apparatus further comprises one or more microphones configured to detect sound, wherein the processing module is configured to determine whether the detected sound is associated with snoring or not.

Optionally, the processing module is configured to provide feedback to the user after determining that the detected sound is associated with the snoring.

Optionally, the feedback has an intensity sufficient to cause the user to stop the snoring.

Optionally, the apparatus further comprises an accelerometer configured to detect an orientation associated with the user.

Optionally, the processing module is configured to monitor a sleeping behavior of the user based on the detected orientation.

Optionally, the processing module comprises a light sensor.

Optionally, the processing module is configured to determine a sleeping parameter based on an output from the light sensor.

Optionally, the processing module comprises a communication unit configured to communicate wirelessly with an external device.

Optionally, the communication unit comprises a wireless device.

Optionally, the communication unit comprises a cable port.

Optionally, the external device is a cell phone, a computer, a server, a tablet, or a watch.

Optionally, the processing module comprises a rechargeable battery, a circuitry.

Optionally, the processing module comprises a housing having a crescent shape.

Optionally, the processing module comprises a non-transitory medium.

Optionally, the non-transitory medium is configured to store a time-series of pressure values outputted by the sensor.

Optionally, the non-transitory medium is configured to store a time-series of pressure threshold values.

Optionally, the non-transitory medium is configured to store sound data recorded during a sleeping event of the user.

Optionally, the apparatus is configured to provide feedback to the user, and the non-transitory medium is configured to store a time at which the feedback is provided to the user.

Optionally, the apparatus is configured to provide feedback to the user if the pressure due to the biting force exceeds a pressure threshold.

Optionally, the pressure threshold has a constant value.

Optionally, the pressure threshold is variable over time.

Optionally, the pressure threshold is variable based on an averaging pressure values associated with a non-bruxing event in a moving temporal window.

Optionally, the apparatus further comprises a temperature sensor, wherein the pressure threshold is based on an output provided by the temperature sensor.

Optionally, the pressure threshold has a value that is larger than a baseline pressure value associated with non-bruxing event.

Optionally, the pressure threshold value is at least 10% larger than the baseline pressure value.

Optionally, the processing module is configured to determine a baseline pressure value associated with a non-bruxing event.

Optionally, the processing module is configured to determine the baseline pressure value by averaging pressure values associated with the non-bruxing event in a moving temporal window.

Optionally, the sensor comprises a pressure sensor configured to detect pressure as the characteristic, the pressure comprising a difference pressure with respect to a baseline pressure, and wherein the pressure sensor is configured to detect the difference pressure with respect to the baseline pressure.

Optionally, the processing module is configured to calculate a pressure change by subtracting a value of the detected pressure from a baseline pressure value.

Optionally, the characteristic comprises pressure.

Optionally, the characteristic comprises force.

Optionally, the characteristic comprises current.

Optionally, the characteristic comprises electrical resistance.

Optionally, the apparatus may be a part of a system that comprises an electronic device, wherein the apparatus and the electronic device are configured to communicate with each other, and wherein the electronic device is configured to: provide a first metric value indicating a level of bite force; allow the user to select whether the user wants the apparatus to provide feedback or not; allow the user to set a feedback threshold value, above which will cause the apparatus to provide the feedback; or a combination of two or more of the foregoing.

A mouthpiece includes: a body comprising one or more portions defining a space for accommodating one or more teeth of a user; and a first cavity at least partly in the body, wherein a volume inside the first cavity is variable in response to a compression applied to the body.

Optionally, the one or more teeth comprise one or more upper teeth, and wherein the space is configured to accommodate the one or more upper teeth.

Optionally, the one or more teeth comprise one or more lower teeth, and wherein the space is configured to accommodate the one or more lower teeth.

Optionally, the mouthpiece further comprises a second cavity, wherein the first cavity is at one of a left side or a right side of the body, and the second cavity is at another one of the left side or the right side of the body.

Optionally, the mouthpiece further comprises a spacer configured for placement in the first cavity or the second cavity.

Optionally, the mouthpiece is a customized mouthpiece.

Optionally, the body comprises at least two layers of EVA materials laminated together.

Optionally, the body is transparent or semi-transparent.

Optionally, the mouthpiece further comprises a first tube coupled to, and/or extending from, the body.

Optionally, the first tube is removably coupled to the body.

Optionally, the first tube is integrally formed with the body.

Optionally, the first tube is on one of a left side or a right side of the body.

Optionally, the mouthpiece further comprises a second tube, wherein the second tube is on another one of the left side or the right side of the body.

Optionally, the cavity is between two or more layers of materials that are laminated together.

Optionally, the one or more layers of materials are parts of the body, and are integral with the body.

Optionally, the one or more layers of materials are attached to the body.

Optionally, the mouthpiece is a 3D-printed mouthpiece.

Optionally, the 3D-printed mouthpiece comprises a biocompatible dental resin.

Optionally, the cavity is positioned to respond to a pressure applied on a molar, a premolar, a canine tooth, or any combination of two or more of the foregoing.

Optionally, the mouthpiece may be a part of an apparatus that includes a connector.

Optionally, the connector comprises a tube; wherein the tube has a first end, a second end opposite from the first end, and a tubular body extending between the first end and the second end; wherein the first end of the tube is configured to couple to the mouthpiece; and wherein the second end of the tube is configured to couple to a processing module.

Optionally, the tube has a stiffness sufficient to transmit a vibrational force from the processing module to the mouthpiece.

Optionally, the processing module comprises a sensor configured to detect a characteristic due to a bruxing event.

Optionally, the characteristic comprises pressure.

Optionally, the characteristic comprises force.

Optionally, the characteristic comprises current.

Optionally, the characteristic comprises electrical resistance.

Optionally, the mouthpiece comprises a processing module.

Optionally, the processing module is configured to determine whether the user is having an apnea event or not, and wherein the apparatus is configured to provide feedback to the user after determining that the user is having the apnea event.

Optionally, the feedback has an intensity sufficient to cause the user to stop the apnea event.

Optionally, the processing module is configured to determine whether the user is having the apnea event or not based on an output from a microphone and/or an output from a pulse oximeter.

Optionally, the mouthpiece comprises a sensor and a feedback signal generator, wherein the cavity is coupled to the sensor.

Optionally, the mouthpiece further comprises a pulse oximeter.

Optionally, the mouthpiece further comprises a temperature sensor.

Optionally, the mouthpiece further comprises an accelerometer.

Optionally, the mouthpiece further comprises a communication device.

Optionally, the mouthpiece further comprises a non-transitory medium storing data associated with an operation of the mouthpiece.

A connector for coupling a mouthpiece to a processing module includes: a tube having a first end, a second end opposite from the first end, and a tubular body extending between the first end and the second end; wherein the first end of the tube is configured to couple to the mouthpiece; wherein the second end of the tube is configured to couple to the processing module; and wherein the tube has a stiffness sufficient to transmit a vibrational force from the processing module to the mouthpiece.

Optionally, the first end of the tube is configured to detachably couple to the mouthpiece.

Optionally, the second end of the tube is configured to detachably couple to the processing module.

Optionally, the tube has a third end configured to couple to the mouthpiece.

Optionally, the first end of the tube is configured to couple to one of a left side or a right side of the mouthpiece, and the third end of the tube is configured to couple to another one of the left side or the right side of the mouthpiece.

Optionally, the first end of the tube is configured to couple to one of a left side or a right side of the mouthpiece.

Optionally, at least a part of the tube is elastically deformable in response to a biting force applied by a user.

An apparatus includes: a housing; and a sensor contained in the housing, the sensor configured to detect a characteristic due to a biting force applied to a mouthpiece by a user.

Optionally, the apparatus further comprises a tube extending from the housing, wherein the tube has an end configured to couple to a mouthpiece.

Optionally, the apparatus further comprises the mouthpiece, wherein the mouthpiece comprises a first cavity configured to be in fluid communication with a lumen of the tube.

Optionally, the apparatus is configured to provide feedback to the user if the pressure due to the biting force exceeds a pressure threshold.

Optionally, the pressure threshold has a constant value.

Optionally, the pressure threshold is variable over time.

Optionally, the pressure threshold is variable based on an averaging pressure values associated with a non-bruxing event in a moving temporal window.

Optionally, the apparatus further comprises a temperature sensor, wherein the pressure threshold is based on an output provided by the temperature sensor.

Optionally, the pressure threshold has a value that is larger than a baseline pressure value associated with a non-bruxing event.

Optionally, the pressure threshold value is at least 10% larger than the baseline pressure value.

Optionally, the apparatus further comprises a processing module configured to determine a baseline pressure value associated with a non-bruxing event.

Optionally, the processing module is configured to determine the baseline pressure value by averaging pressure values associated with the non-bruxing event in a moving temporal window.

Optionally, the moving temporal window has a duration of 5 minutes or less.

Optionally, the sensor is a part of a processing module.

Optionally, the sensor comprises a pressure sensor configured to detect pressure as the characteristic, the pressure comprising a difference pressure with respect to a baseline pressure, and wherein the pressure sensor is configured to detect the difference pressure with respect to the baseline pressure.

Optionally, the apparatus is configured to calculate a pressure change by subtracting a value of the detected pressure from a baseline pressure value.

Optionally, the apparatus further comprises a feedback signal generator configured to provide feedback to the user.

Optionally, the feedback signal generator comprises a vibrational force generator configured to provide vibration energy as the feedback.

Optionally, the feedback signal generator comprises a sound generator configured to provide an audio output as the feedback.

Optionally, the feedback signal generator comprises a light generator configured to provide light as the feedback.

Optionally, the feedback signal generator comprises a current generator configured to provide an electric signal as the feedback.

Optionally, the feedback has an intensity sufficient to cause the user to stop or reduce the biting force.

Optionally, the intensity of the feedback is based on a failure or a success of a previous feedback to cause a reaction from the user.

Optionally, the apparatus is configured to detect a snoring of the user, and wherein the feedback has an intensity sufficient to cause the user to stop the snoring.

Optionally, the apparatus further comprises one or more microphones configured to detect sound, wherein the apparatus is configured to determine whether the detected sound is associated with snoring or not.

Optionally, the apparatus is configured to provide feedback to the user after determining that the detected sound is associated with the snoring.

Optionally, the feedback has an intensity sufficient to cause the user to stop the snoring.

Optionally, the apparatus further comprises an accelerometer configured to detect an orientation associated with the user.

Optionally, the apparatus is configured to monitor a sleeping behavior of the user based on the detected orientation.

Optionally, the apparatus further comprises a light sensor.

Optionally, the apparatus is configured to determine a sleeping parameter based on an output from the light sensor.

Optionally, the apparatus further comprises a communication unit configured to communicate wirelessly with an external device.

Optionally, the communication unit comprises a wireless device.

Optionally, the communication unit comprises a cable port.

Optionally, the external device is a cell phone, a computer, a server, a tablet, or a watch.

Optionally, the apparatus further comprises a rechargeable battery contained by the housing.

Optionally, the housing has a crescent shape.

Optionally, the apparatus further comprises a non-transitory medium contained by the housing.

Optionally, the non-transitory medium is configured to store a time-series of pressure values outputted by the sensor.

Optionally, the non-transitory medium is configured to store a time-series of pressure threshold values.

Optionally, the non-transitory medium is configured to store sound data recorded during a sleeping event of the user.

Optionally, the apparatus is configured to provide feedback to the user, and the non-transitory medium is configured to store a time at which the feedback is provided to the user.

Optionally, the apparatus further comprises a pulse oximeter.

Optionally, the housing is in the mouthpiece.

Optionally, the housing is detachably coupled to the mouthpiece, and wherein the housing is configured for placement inside a mouth of the user.

Optionally, the housing is the mouthpiece, or is implemented as a part of the mouthpiece.

An electronic device for communication with an apparatus having a mouthpiece, includes: a transceiver configured to communicate with the apparatus; and a screen configured to display information to a user of the apparatus; wherein the electronic device is configured to: provide a first metric value indicating a level of bite force applied at the mouthpiece; allow the user to select whether the user wants the apparatus to provide feedback or not; allow the user to set a feedback threshold value, above which will cause the apparatus to provide the feedback; or a combination of two or more of the foregoing.

Other and further aspects and features will be evident from reading the following detailed description.

### BRIEF DESCRIPTION OF THE DRAWINGS

The drawings illustrate the design and utility of embodiments, in which similar elements are referred to by common reference numerals. In order to better appreciate how advantages and objects are obtained, a more particular description of the embodiments will be described with reference to the accompanying drawings. Understanding that these drawings depict only exemplary embodiments and are not therefore to be considered limiting in the scope of the claimed invention.

FIG. 1 illustrates an apparatus having a mouthpiece, a processing module, and a tube.

FIG. 2 illustrates the apparatus of FIG. 1, particularly showing the apparatus coupled to a charging/communication cable.

FIG. 3 illustrates the processing module of the apparatus of FIG. 1.

FIG. 4 illustrates exemplary components of the processing module of FIG. 1.

FIG. 5 illustrates an example of the mouthpiece of FIG. 1.

FIG. 6 illustrates a system that includes the apparatus of FIG. 1 and an electronic device.

FIG. 7 illustrates an example of a circuit diagram for a circuit in the processing module of FIG. 1.

FIGS. 8A-8C illustrate other exemplary circuit diagrams in the processing module of FIG. 1.

FIG. 9 illustrates the apparatus of FIG. 1, about to be inserted into a mouth of a user.

FIG. 10 illustrates the apparatus of FIG. 1, with the mouthpiece being engaged with teeth of the user.

FIG. 11 illustrates the apparatus of FIG. 1, with the mouthpiece being inside the mouth of the user.

FIG. 12 illustrates a variation of the apparatus of FIG. 1, particularly showing the tube extending from a long side of the processing module.

FIG. 13A-FIG. 13B illustrate the tube of the apparatus of FIG. 12 being coupled to the mouthpiece of the apparatus.

FIG. 14A-FIG. 14B illustrate the apparatus of FIG. 12 being coupled to a charge/communication cable.

FIG. 15 illustrates an example of the mouthpiece in the apparatus of FIG. 1 or FIG. 12.

FIG. 16 illustrates an example of the tube in the apparatus of FIG. 1 or FIG. 12.

FIG. 17 illustrates an example of an electronic device configured to communicate with the apparatus of FIG. 1 or FIG. 12.

FIG. 18 illustrates a pressure testing apparatus configured to obtain pressure reading of the apparatus of FIG. 1 or FIG. 12.

FIG. 19 illustrates a vibration testing apparatus configured to test a vibrational force provided by the apparatus of FIG. 1 or FIG. 12.

FIG. 20 illustrates exemplary components in the processing module of the apparatus of FIG. 12.

FIG. 21 illustrates exemplary components of the apparatus of FIG. 12.

FIG. 22 illustrates example of data captured by the apparatus of FIG. 1 or FIG. 12, particularly showing the data having metrics indicating levels of bite force over time.

FIG. 23 illustrates example of data captured by the apparatus of FIG. 1 or FIG. 12, particularly showing the data having metrics indicating levels of bite force over time, threshold values for triggering feedback, and timepoints at which feedback was provided.

FIG. 24 illustrates a component having multiple microphones, wherein the component is configured to differentiate breathing sound from snoring sound.

FIG. 25 illustrates microphone outputs from the component of FIG. 24.

FIG. 26 illustrates data generated by the component of FIG. 24, particularly showing the component being capable of differentiating breathing sound from snoring sound.

FIG. 27 illustrates another example of the processing module of FIG. 1.

FIG. 28 illustrates another example of the tube of FIG. 1.

FIG. 29 illustrates the tube of FIG. 28 being coupled with the processing module of FIG. 27.

FIG. 30 illustrates a variation of the apparatus of FIG. 1.

FIGS. 31-39 illustrate another variation of the apparatus of FIG. 1.

### DESCRIPTION OF THE EMBODIMENTS

Various embodiments are described hereinafter with reference to the figures. It should be noted that elements of similar structures or functions are represented by like reference numerals throughout the figures. It should also be noted that the figures are only intended to facilitate the description of the embodiments. They are not intended as an exhaustive description of the claimed invention or as a limitation on the scope of the claimed invention. In addition, an illustrated embodiment needs not have all the aspects or advantages of the invention shown. An aspect or an advantage described in conjunction with a particular embodiment is not necessarily limited to that embodiment and can be practiced in any other embodiments even if not so illustrated or if not so explicitly described.

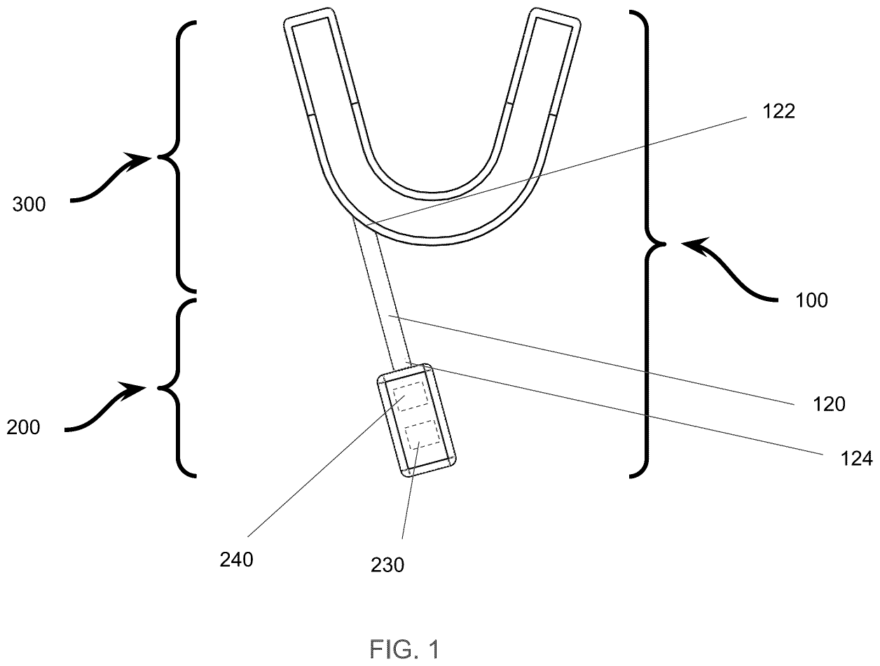

FIG. 1 illustrates an apparatus 100 in accordance with some embodiments. The apparatus 100 has a mouthpiece 300, a processing module 200, and a connector 120. The mouthpiece 300 is configured for placement inside a mouth of a user during use. The connector 120 connects the mouthpiece 300 and the processing module 200 to each other. The connector 120 is a tube having a first end 122 coupled to the mouthpiece, and a second end 124 coupled to the processing module 200. The processing module 200 comprises a pressure sensor 230 configured to detect a pressure due to a biting force applied to the mouthpiece 300 by the user. The processing module 200 also comprises a feedback signal generator 240 configured to provide a feedback in response to the detected pressure satisfying a criterion. The processing module 200 may be waterproof in some embodiments.

In the illustrated embodiments, the connector 120 is a tube with a lumen. The tube 120 is configured to connect the mouthpiece 300 and the processing module 200, so that the lumen of the tube 120 is in fluid communication with a cavity in the mouthpiece 300. During use, when the user applies a biting force to the mouthpiece 300, the cavity and/or the first end 122 of the tube 120 in the mouthpiece 300 will deform to cause an increase of pressure inside the lumen of the tube 120. The pressure sensor 230 of the processing module 200 is configured to detect the pressure inside the lumen of the tube 120, and to determine whether a criterion is satisfied based on the detected pressure. In some cases, if the processing module 200 determines that the criterion is satisfied, the processing module 200 then determines that there is a bruxing event.

In the illustrated embodiments, if the processing module 200 of the apparatus 100 determines that the criterion is satisfied based on the detected pressure, the feedback signal generator 240 in the processing module 200 then generates feedback for the user. The feedback may have an intensity sufficient to cause the user to stop or reduce the biting force being applied to the mouthpiece 300.

In some cases, the feedback signal generator 240 may comprise a vibrational force generator (e.g., motor) configured to provide vibration energy as the feedback, and wherein the tube 120 is configured to transmit the vibration energy to the user. Alternatively, the feedback signal generator 240 may comprise a sound generator configured to provide an audio output as the feedback. Alternatively, the feedback signal generator 240 may comprise a light generator configured to provide light as the feedback. Alternatively, the feedback signal generator 240 may comprise a current generator configured to provide an electric signal (e.g., electrical shock) as the feedback. In other cases, the feedback signal generator 240 may be a heater configured to provide a certain temperature (e.g., heat) to the user as feedback. In further cases, the feedback signal generator 240 may be an air blower configured to provide air puff(s) as feedback for the user. Also, in further cases, the feedback signal generator 240 may be configured to provide multiple types of feedback, such as two or more of: vibration energy, audio output, light, or electric signal, temperature, air puff (air force), etc. One or more of the above feedbacks may be provided by the apparatus 100 to one or more anatomies of the user, such as to the mouth, gum(s), teeth, lip, cheek, a buccal anatomy, etc.

In some cases, the apparatus 100 is configured to provide feedback to the user if the pressure due to the biting force exceeds a pressure threshold (which is an example of a criterion). In one implementation, the pressure threshold has a constant value. In an alternative implementation, the pressure threshold is variable over time. For example, the pressure threshold may be variable based on an averaging pressure values associated with a non-bruxing event in a moving temporal window. The moving temporal window may have a duration, such as five minutes or less.

In some cases, the pressure threshold may have a value that is larger than a baseline pressure value associated with non-bruxing event. For example, the pressure threshold value may be at least 10% (e.g., 10%, 15%, 20%, 25%, etc.) larger than the baseline pressure value. Various techniques may be employed to determine the baseline pressure value. In some cases, the baseline pressure value may be determined statistically be collecting data from a population. For example, different baseline pressure values may be determined for different age groups and/or genders. During use, the processing module 200 may be configured to set a desired baseline pressure value based on the age of the user and/or the gender of the user.

In another technique, the processing module 200 may be configured to determine a baseline pressure value associated with a non-bruxing event based on usage of the apparatus 100 by the user. For example, the processing module 200 may be configured to determine the baseline pressure value by averaging pressure values associated with the non-bruxing event in a moving temporal window. In one implementation, while the user is using the apparatus 200, the sensor 230 repeatedly detects pressure due to biting force applied to the mouthpiece 300 by the user, and provides pressure values for processing by a processing unit in the processing module 200. The processing unit may calculate the average of the pressure values generated in a moving temporal window (e.g., last 5 seconds, last 10 seconds, last 30 seconds, etc.) as the baseline pressure value. Also, in some embodiments, the processing module 200 may determine the baseline pressure value. or a metric value indicating a level of biting, by receiving such value transmitted by an electronic device (such as the electronic device 500 described herein). The pressure threshold may then be determined based on the baseline pressure value.

In some embodiments, the processing module 200 may be configured to update the threshold based on a fixed frequency, e.g., every 30 seconds, every 1 minute, every 2 minutes, etc. In other embodiments, the processing module 200 may be configured to update the threshold every sensor sample, every other sensor sample, or every x-number of sensor samples (where x may be 2 or higher).

The updating of the threshold is advantageous because a bite signal may change over time as the mouthpiece 300 and/or the tube 120 wears or deteriorates. The updating also accounts for the difference in anatomy, biting mechanics, and interaction of the tube 120 with the profile of the user's teeth, that are different from person to person, thereby providing different thresholds for different users. The updating may also compensate for the situation when the tube 120 is coupled properly with (e.g., not fully inserted into) the mouthpiece 300.

In some cases, the intensity of the feedback provided by the apparatus 100 is constant. In other cases, the intensity of the feedback provided by the apparatus 100 may be variable. For example, the intensity of the feedback may be based on a failure or a success of a previous feedback to cause a reaction from the user. If a previous feedback fails to cause a reaction by the user (e.g., the user fails to relax the jaw to stop a bruxing event), the processing module 200 may then increase the intensity of the feedback until a reaction criterion is achieved. For example, the reaction criterion may be considered as achieved if the detected pressure by the pressure sensor 230 is less than a pressure threshold. In other cases in which a different type of sensor is used, the reaction criterion may be considered as achieved if the detected parameter by the sensor is less than a parameter threshold.

Optionally, the apparatus 100 may further include one or more temperature sensors, wherein the pressure threshold is based on an output provided by the temperature sensor(s). A person's body temperature may change slightly throughout a night's sleep. Thus, the air in the tube 120 will increase with increasing body temperature, and will decrease with decreasing body temperature. To compensate for this, the processing module 200 may use temperature value provided by the temperature sensor to adjust a sensed pressure by the pressure sensor. In one implementation, the temperature sensor may be attached to the tube (connector) 120, such as anywhere along the tube body, or at the first end 122 or the second end 124 of the tube 120. In other cases, the temperature sensor may be located in the processing module 200 or may be attached to an exterior of the processing module 200. In some cases, the pressure threshold for evaluating whether to provide feedback may be determined based on certain temperature or assumption of temperature. In such cases, when the user is using the apparatus 100, the sensed pressure may be for a temperature that is different from the temperature associated with the pressure threshold. The processing module 200 may be configured to adjust the sensed pressure based on the equation P1/T1=P2/T2, where P1 is the sensed pressure, T1 is the temperature corresponding with the sensed pressure P1, P2 is the adjusted pressure, and T2 is the temperature corresponding with the adjusted pressure P2. Thus, P2 (the adjusted pressure) may be determined by the processing unit 200 as P2=P1*T2/T1.

Also, in some cases, the sensor output (e.g., temperature data) from the temperature sensor(s) may be used by the processing module 200 to determine whether the apparatus is in use (e.g., when the mouthpiece 300 is inside the mouth) or not. The processing module 200 may also log and track the timing at which the apparatus 100 is in use based on the sensor output (e.g., temperature data).

In other cases, the temperature sensor is not needed, and the apparatus 100 may not include the temperature sensor. For example, the pressure threshold for providing feedback may have a corresponding temperature that is about the same as the temperature associated with sensed pressure by the pressure sensor 230. This may happen when the pressure threshold is based on an average of pressure values obtained in the moving temporal window as described herein. Thus, the pressure threshold is based of sensed pressure values obtained the same way as pressure values are obtained during use of the apparatus 100.

In some cases, the pressure sensor 230 is configured to detect the pressure within the lumen of the connector 120. In other cases, the pressure detected by the sensor 230 may be a difference pressure with respect to a baseline pressure. In further cases, the processing module 200 may be configured to calculate a pressure change by subtracting a value of the detected pressure from a baseline pressure value.

In the illustrated example, the apparatus 100 is described as having pressure sensor 230 configured to sense pressure that is associated with the event of the user applying biting force. In other cases, item 230 may not be a pressure sensor 230, and may instead be any of other types of sensor configured to detect a characteristic due to a biting force applied to the mouthpiece 300 by the user. For example, in other cases, item 230 may be a force sensor configured to detect force due to a biting event applied to the mouthpiece 300. Alternatively, item 230 may be a current sensor configured to detect a current due to a biting event applied to the mouthpiece 300. In further cases, item 230 may be an electrical resistance sensor configured to detect an electrical resistance due to a biting event applied to the mouthpiece 300. Thus, the sensor 230 may be different types of sensors in different embodiments.

If any of these other types of sensors is used instead of the pressure sensor, the threshold for determining whether to provide feedback to the user may be determined using a similar technique as that described previously. In particular, the processing module 200 may be configured to determine a baseline metric value associated with a non-bruxing event based on usage of the apparatus 100 by the user. For example, the processing module 200 may be configured to determine the baseline metric value by averaging metric values associated with the non-bruxing event in a moving temporal window. In one implementation, while the user is using the apparatus 200, the sensor 230 repeatedly detects a characteristic due to biting force applied to the mouthpiece 300 by the user, and provides sensor values for processing by a processing unit in the processing module 200. The processing unit may calculate the average of the sensor values generated in a moving temporal window (e.g., last 5 seconds, last 10 seconds, last 30 seconds, etc.) as the baseline metric value. Also, in some embodiments, the processing module 200 may determine the baseline metric value. or a metric value indicating a level of biting, by receiving such value transmitted by an electronic device (such as the electronic device 500 described herein). The threshold for determining whether to provide feedback may then be determined based on the baseline metric value. For example, the threshold may be set to be a certain percentage above the baseline metric value.

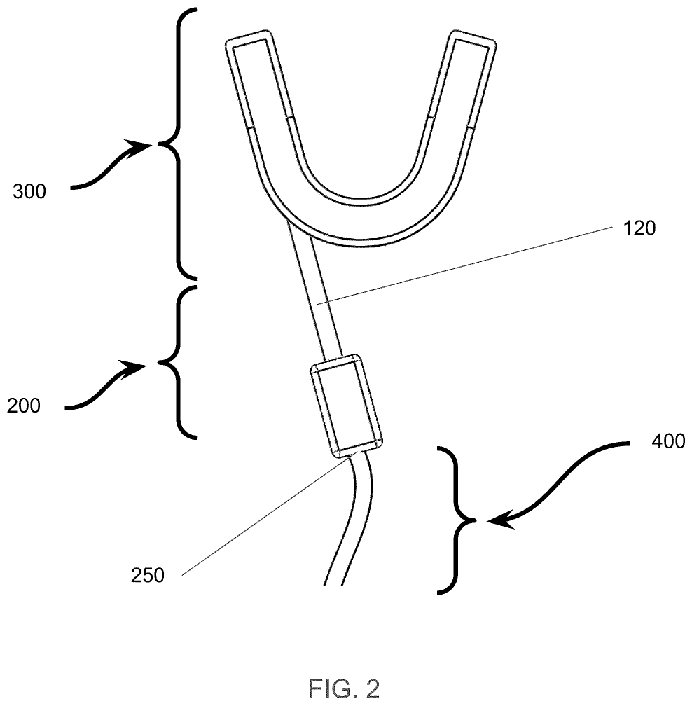

In some cases, the processing module 200 may include an electrical port configured to receive a charging/communication cable. FIG. 2 illustrates the apparatus 100 of FIG. 1, particularly showing the apparatus 100 coupled to a charging/communication cable 400. As shown in the figure, the processing module 200 of the apparatus 100 includes an electrical port 250 configured to receive the charging/communication cable 400. The electrical port 250 may be configured to receive charging power from an outlet via the cable 400, and/or to communicate with an electronic device via the cable 400. For example, in some cases, the electrical port 250 of the processing module 200 may be configured to receive configuration data from the electronic device to configure the processing module 200. In another example, the electrical port 250 of the processing module 200 may be configured to transmit data to the electronic device, such as data collected by the processing module 200 during use of the apparatus 100.

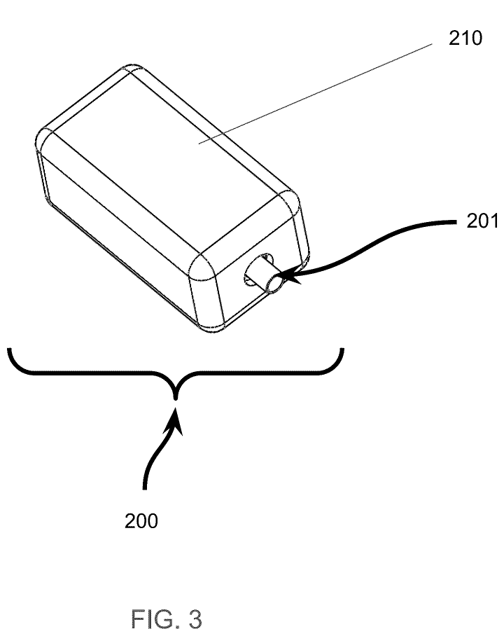

FIG. 3 illustrates the processing module 200 of the apparatus 100 of FIG. 1. As shown in the figure, the processing module 200 includes a housing 210, and a port 201 configured to couple to the second end 204 of the connector (tube) 120. In the illustrated example, the second end 204 of the tube 120 is configured to detachably couple to the processing module 200 via the port 201. The detachably coupling may be implemented using a snap-fit connector, a screw-connector, a frictional-fit connector, or any of other types of connectors. In other cases, the second end 204 of the tube 120 may be fixedly and permanently attached to the processing module 200. In some cases, the port 201 may be a part of the pressure sensor 230.

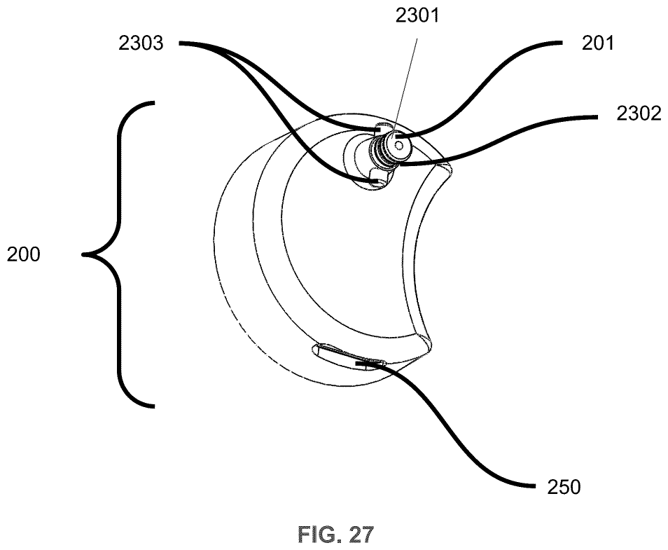

Also, in some cases, the processing module 200 may include a locking mechanism configured to lock the second end 204 of the tube 120 with respect to the processing module 200. The locking mechanism may be any mechanical lock (such as that shown in FIG. 27, a screw-type lock, a locking pin, an anchor, etc.). The locking mechanism is advantageous because it prevents the tube 120 from accidentally detached from the processing module 200, thereby preventing, or reducing the risk of, the user swallowing the mouthpiece 300. In other cases, the locking mechanism is not needed. In such cases, even if the tube 120 is accidentally detached from the processing module 200, the mouthpiece 300 with the tube 120 may still be difficult to swallow, which provides some level of safety for the user.

Similarly, in some cases, the mouthpiece 300 may include a locking mechanism configured to lock the first end 202 of the tube 120 with respect to the mouthpiece 300. The locking mechanism may be any mechanical lock (such as a screw-type lock, a locking pin, an anchor, etc.). The locking mechanism is advantageous because it prevents the tube 120 from accidentally detached from the mouthpiece 300, thereby preventing, or reducing the risk of, the user swallowing the mouthpiece 300 together with the tube 120. In other cases, the locking mechanism is not needed.

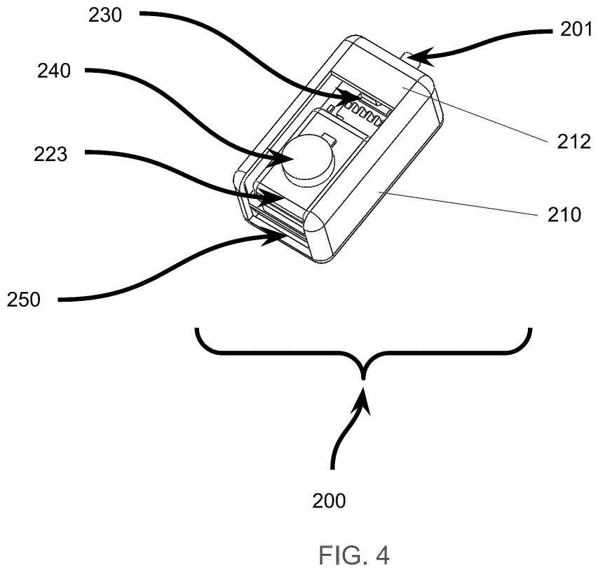

FIG. 4 illustrates exemplary components of the processing module 200 of FIG. 1. As shown in the figure, the processing module 200 includes the housing 210 defining a housing cavity 212, the feedback signal generator 240, a battery 223, a pressure sensor 230, and the electrical port 250. The feedback signal generator 240, the pressure sensor 230, and the battery 223 are accommodated in the housing cavity 212 of the housing 210. In the illustrated example, the feedback signal generator 240 is a vibrational force generator. The vibrational force generator is configured to generate vibration as feedback in response to the pressure sensed by the pressure sensor 230 exceeding a pressure threshold. The battery 223 may be a rechargeable battery, or may be a non-rechargeable battery. The battery 223 supplies power for the pressure sensor 230 and the feedback signal generator 240. If the battery 223 is a rechargeable battery 223, the battery 223 may be recharged via the electrical port 250 of the processing module 200.

In some cases, the processing module may optionally further include a communication unit configured to communicate with an external device. The communication unit may be a wireless device configured to communicate wirelessly with the external device. Alternatively, the communication unit may be the electrical port 250 configured to communicate with the external device via a cable. The external device may a cell phone, a computer, a server, a tablet, a watch, or any of other devices.

Also, in some cases, the pressure sensor 230 and the feedback signal generator 240 may be considered as parts of a processing unit that is accommodated in the housing 210 of the processing module 200. The processing unit may be implemented using hardware, software, or a combination of both hardware and software. In some cases, the processing unit may include one or more processors and/or an integrated circuit configured to implement one or more functionalities described herein.

In the illustrated example of FIG. 4, the housing 210 has a rectangular shape. In other cases, the housing 210 may have other shapes. For example, in other embodiments, the housing the housing 210 may have a crescent shape.

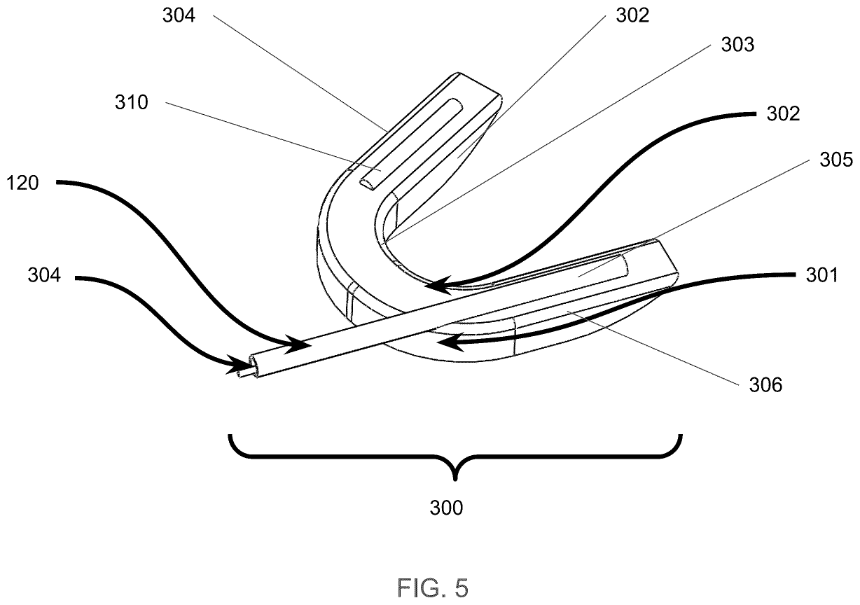

FIG. 5 illustrates an example of the mouthpiece 300 in the apparatus 100 of FIG. 1. The mouthpiece 300 includes an inner wall 302, an outer wall 304, and a bite portion 306 between the inner wall 302 and the outer wall 304. The mouthpiece 300 also includes a first cavity 305 configured to be in fluid communication with the lumen of the tube (connector) 120. The first cavity 305 is on one of a left side or a right side of the mouthpiece 300. The first cavity 305 located at the bite portion 306 of the mouthpiece 300 so that when a user applies biting force (as during bruxing event), the first cavity 305 will be compressed to reduce the volume of the first cavity 305. Thus, the first cavity 305 is variable in response to a compression applied to the mouthpiece 300.

In the illustrated example, the inner wall 302, the outer wall 304, and the bite portion 306 are parts of a body 303 of the mouthpiece 300. The body 303 of the mouthpiece 300 comprises one or more portions (e.g., the inner wall 302 and/or the outer wall 304) defining a space for accommodating one or more teeth of a user. In some cases, the space of the mouthpiece 300 may accommodate one or more upper teeth. In other cases, the space of the mouthpiece 300 may accommodate one or more lower teeth. In further cases, the space of the mouthpiece 300 may accommodate both upper and lower teeth of the user. The body of the mouthpiece 300 may be transparent, semi-transparent, or opaque.

In some cases, the mouthpiece 300 may comprise at least two layers of materials laminated together. The first cavity 305 may be between two or more layers of materials that are laminated together. In the illustrated example of FIG. 5, the mouthpiece 300 includes a first layer 301 and a second layer 302 coupled to each other to form a laminated structure. The first layer 301 is more rigid than the second layer 302. For example, the first layer 301 may be a hard splint configured to interface with the teeth of the user, and the second layer 302 may be a soft and deformable layer. The second layer 302 may be made from a polymeric material, such as PVA. In some cases, the one or more layers of materials form the body 303 of the mouthpiece 300. In other cases, the one or more layers of materials are parts of the body 303, and are integral with the body 303. In further cases, the one or more layers of materials are attached to the body 303 of the mouthpiece 300.

In some cases, the mouthpiece 300 may optionally further include a spacer 310 at the opposite side of the mouthpiece 300 from the first cavity 305. The spacer 310 may have a thickness (height) that is substantially the same (e.g., with difference in dimension that is +/−10%) as that of the first cavity 305, so that both the left and right teeth of the user will simultaneously engage with the mouthpiece 300 when the user applies biting force. The first cavity 305 may be on the left side of the mouthpiece 300, and the spacer 310 may be at the opposite side (the right side) of the mouthpiece 300. In other cases, the first cavity 305 may be on the right side of the mouthpiece 300, and the spacer 310 may be at the opposite side (the left side) of the mouthpiece 300.

As shown in the figure, the tube (connector) 120 is attached to the mouthpiece 300. The tube 120 may be made from a polymer, plastic, elastomer (e.g., silicone), or any of other suitable materials. In some cases, the material of the tube 120 may be the same as at least a part of the mouthpiece 300. The tube 120 may be a separate component from the mouthpiece 300, and is detachably attached to the mouthpiece 300. Alternatively, the tube 120 and the mouthpiece 300 may be integrally formed together so that they form an unity configuration. In such cases, the tube 120 and the mouthpiece 300 are permanently connected to each other. In the illustrated example, the first end 122 of the tube 120 is inserted into an opening at the mouthpiece 300 and is advanced until the lumen of the tube 120 is in fluid communication with the first cavity 305.

In some cases, the first end 122 of the tube 120 may be inserted all the way to an end (e.g., the proximal end) of the first cavity 305. In such cases, when the user applies biting force to compress the first cavity 305, the biting force will also compress a part of the tube 120 that is in the first cavity 305. In other cases, the first end 122 of the tube 120 may be inserted until it reaches a distal end of the first cavity 305 (i.e., without reaching the proximal end of the first cavity 305). In such cases, when the user applies biting force, the first cavity 305 will deform to reduce its size in response to the biting force, but the tube 120 will not deform.

In the illustrated embodiments, the first cavity 305 is defined between two or more layers of the mouthpiece 300. In other embodiments, the first cavity 305 may be surrounded or defined by an air bladder, which is sandwiched between two or more layers of the mouthpiece 300. The air bladder may be made from silicone, a polymer, or any of other suitable materials. In some cases, the first cavity 305 may be implemented using a tube with a first closed end, and an opposite open end. The tube may be sandwiched between two layers of materials of the mouthpiece 300. Alternatively, the tube may be secured on (e.g., integrally formed with) an exterior surface of the body 303 of the mouthpiece 300.

In some cases, the mouthpiece 300 is a customized mouthpiece. For example, a dentist or a technician may scan the inner mouth (e.g., the teeth) of the user, or obtain a mold of the inner mouth (e.g., the teeth) of the user, and provide the scanned data or the mold to a manufacturer of the mouthpiece 300. In other cases, the mouthpiece 300 is a non-customized mouthpiece. For example, the mouthpiece 300 may be one of a plurality of standard mouthpieces 300 with different corresponding sizes provided to the user for selection. During a fitting procedure, the user selects one of the standard mouthpieces 300 that is most suitable for the user.

Also, in some cases, the mouthpiece may a 3D-printed mouthpiece. For example, the 3D-printed mouthpiece may be printed from a biocompatible dental resin.

Furthermore, in some cases, the cavity 305 may be positioned to respond to a pressure applied on a molar, a premolar, a canine tooth, or any combination of two or more of the foregoing.

Also, in some cases, the mouthpiece 300 may have a tubular structure extending out of the mouth of the user when the mouthpiece 300 is being used. The tubular structure has an opening at its distal end, and the distal end of the tubular structure is configured to couple to the tube (connector) 120. The tubular structure may be integral with a remaining part of the mouthpiece 300. In other cases, the mouthpiece 300 does not include the tubular structure, and the tube (connector) 120 is coupled to the mouthpiece 300 by inserting into an opening at a part of the mouthpiece 300 that is inside the mouth of the user.

The tube (connector) 120 provides several features for the apparatus 100. The tube (connector) 120 connects the mouthpiece 300 to the processing module 200, maintains the processing unit 200 outside the user's mouth at a fixed positional relation with respect to the mouthpiece 300, provides pressure to the pressure sensor 230 of the processing module 200 during a biting event, and transmits feedback signal from the processing module 200 to the mouthpiece 300 for sensing by the user. Thus, the tube (connector) 120 has sufficient longitudinal length to maintain the processing unit 200 outside the mouth of the user while the mouthpiece 300 is inside the mouth of the user. In the case in which the feedback signal is a mechanical vibration, the tube (connector) 120 should have sufficient stiffness (rigidity) for allowing the transfer of the mechanical vibration. The tube (connector) 120 should also have sufficient stiffness so that the tube can maintain the processing module 200 outside the user's mouth at a fixed position relative to the mouthpiece 300.

As shown in FIG. 5, the tube (connector) 120 may include a stiffener 304 configured to provide the tube 120 with the desirable stiffness. The stiffener 304 may be embedded within a wall of the tube 120, or may be secured to a surface (e.g., an inner surface, or an exterior surface) of the wall of the tube 120. Alternatively, the tube 120 may be configured to have the desirable stiffness, and may not include the stiffener 304. For example, the tube 120 may be made from certain materials having certain desired Modulus of elasticity, and/or the tube 120 may be dimensioned to have certain cross-sectional width and/or wall thickness so that the stiffness of the tube 120 can be achieved. The tube 120 may be made from silicone, polymer, plastic, or any of other suitable materials. In some cases, the outer dimension (e.g., outer diameter) of the tube 120 may be anywhere from 1 mm to 5 mm, or anywhere from 2 mm to 4 mm (e.g., 3 mm). Also, in some cases, the wall thickness of the tube 120 may be anywhere from 0.05 mm to 0.15 mm, or anywhere from 0.08 mm to 0.12 mm (e.g., 0.1 mm). The elastic modulus of the material of the tube 120 may be anywhere from 0.5 GPa to 6 GPa. In other cases, the elastic modulus of the material of the tube 120 may be less than 0.5 GPa. For example, if a stiffening element is coupled to the tube 120, the tube may be made from a material having a relatively lower elastic modulus. The wall thickness of the tube 120 and/or the material of the tube 120 may be selected to provide user comfort.

In some cases, tube 120 may have a circular cross-section. In other cases, the tube 120 may have a non-circular cross-section, such as an oval cross-section, a rectangular cross-section, etc. In some embodiments, the cross-section of the tube 120 may have a first dimension, and a second dimension measured perpendicularly to the first dimension, wherein the second dimension is different from the first dimension. Such permits indexing relative to mouthpiece 300 to ensure that the processing module 200 is orientated correctly with respect to the mouthpiece 300. In some embodiments in which the apparatus 100 includes one or more accelerometers at the tube 120 and/or the processing module 200, the indexing also ensures that the accelerometer(s) is oriented correctly so that accelerometer output may be interpreted correctly by the processing module 200.

In the illustrated example, the tube 120 has a single lumen. In other cases, the tube 120 may have multiple lumens. The lumens of the tube 120 may be communicatively coupled with respective cavities 305 at the mouthpiece 300, thereby allowing biting forces applied at different parts of the mouthpiece 300 to be transmitted to the processing module 200. For example, the tube (connector) 120 may have two lumens. In such cases, a first lumen of the tube 120 may transmit pressure from the left side of the mouthpiece 300, and a second lumen of the tube 120 may transmit pressure from the right side of the mouthpiece 300. As another example, the tube 120 may have four lumens configured to transmit pressure from the left front, the left back, the right front, and the right back of the mouthpiece 300. The processing module 200 may have multiple pressure sensors configured sense the pressure provided by the respective lumens of the tube 120. The processing module 200 may be configured to detect pressure difference and/or pressure changes over time among the different sensing areas (cavities 305) at the mouthpiece 300, and to characterize a grinding pattern of the user based on the detected pressure difference and/or pressure changes over time.

It should be noted that the apparatus 100 should not be limited to having the above features/configurations described, and that the apparatus 100 may have other features and/or configurations in other embodiments. For example, in some embodiments, the processing module 200 may be configured to determine clenching, a degree of clenching, grinding, a degree of grinding, or any combination of the foregoing, based on sensor output from one or more sensors. For example, if sensor output from the sensor 230 indicates that the user applies biting force about a threshold, and the duration of the biting force exceeds a certain duration, then the processing module 200 may determine that a clenching event is occurring. The processing module 200 may also determine a degree of the clenching based on the time duration of the clenching and/or am amplitude of the biting force.

In some embodiments, the mouthpiece 300 may be configured for bilateral biting detection. In such cases, the processing module 200 may include two sensors 230 configured to detect respective force/pressure applied to the left and right sides of the mouthpiece 300. If the sensor outputs indicates that the left and right biting is alternating, then the processing module 200 may determine that a teeth-grinding event is occurring. The processing module 200 may also determine a degree of the teeth-grinding based on the time duration of the teeth-grinding and/or am amplitude of the grinding force.

Also, in other embodiments, the apparatus 100 may optionally further include one or more microphones configured to detect sound, and the processing module 200 is configured to determine whether the detected sound is associated with snoring or not. If the processing module 200 determines that the detected sound is associated with the snoring, the processing module 200 may the provide feedback to the user. The feedback may have an intensity sufficient to cause the user to stop the snoring. In some cases, this may address sleep apnea if the snoring is due to, or associated with, sleep apnea. In particular, the feedback may cause the user to stop the apnea episode.

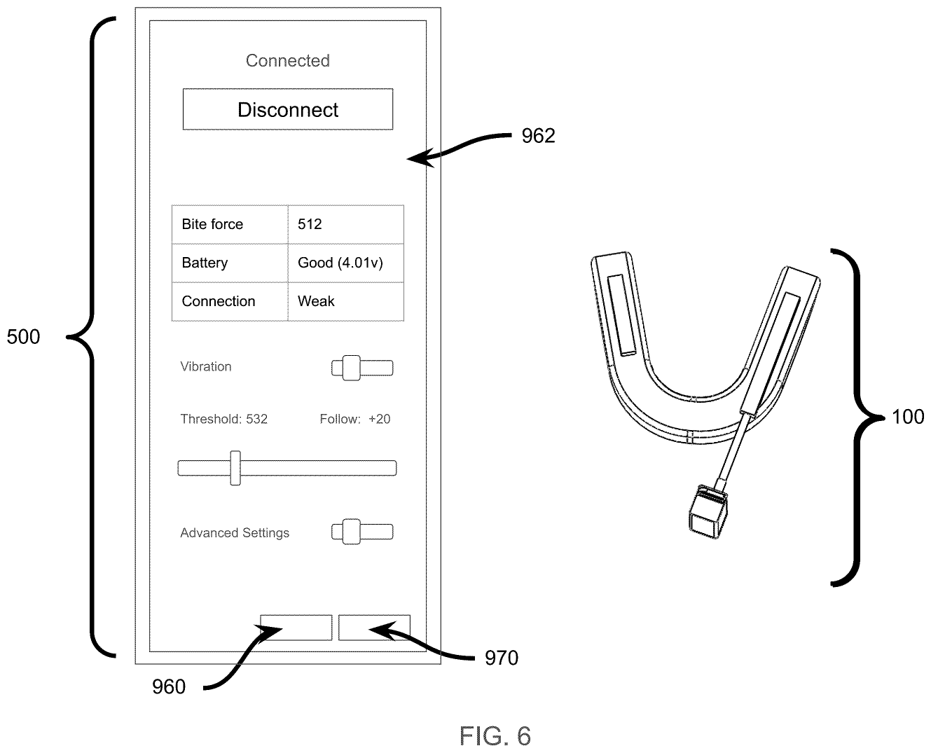

In some cases, the apparatus 100 may optionally further include one or more accelerometers configured to detect an orientation associated with the user. In such cases, the processing module 200 is configured to monitor a sleeping behavior of the user based on the detected orientation. In some embodiments, the accelerometer(s) may detect head orientation. In such cases, the detected head orientation may be utilized by the processing module 200 to determine one or more sleep metrics. For example, the processing module 200 may determine an average frequency of head-turns, a timing of head-turn, etc., based on the detected head orientation. Such metrics may be used by the processing module 200 to determine whether the user has sleeplessness, and to determine a sleep quality measurement for the user. Furthermore, in some cases, the data from the sensors may be utilized by the processing module 200 or an electronic device (such as the electronic device 500 described with reference to FIG. 6) to help the user sleep better. For example, if the processing module 200 or the electronic device detects that the user sleeps more soundly on his/her back, the processing module 200 or the electronic device may make such recommendations to the user so that she/he sleep more on the back. Furthermore, the processing module 200 or the electronic device may suggest products for the user, such as, specific pillows, mattress, bolsters, etc. to help achieve a better sleep for the user.

Also, in some cases, the processing module 200 may optionally include a light sensor (e.g., ambient light sensor). In such cases, the processing module 200 may be configured to determine a sleeping parameter based on an output from the light sensor. For example, the light sensor may detect when the user turns off room lights (presumably before going to sleep), and/or may detect light from electronic devices with screens such as smart phones. The processing module 200 may then determine one or more sleep metrics based on the detected light and related sensing data (e.g., timing of detected light, timing of light being turned off, duration of light-on, etc.). For example, the processing module 200 may determine when the user went to bed, whether electronic device was used before bed time, duration of usage of electronic device before bed time, etc.

In some cases, the processing module 200 may be configured to detect movement of the lower jaw relative to the upper jaw. This may be achieved by implementing a plurality of cavities 305 at the mouthpiece 300. For example, the mouthpiece 300 may have a first cavity towards the incisor teeth, and a second cavity towards the molars. The tube 120 may have two lumens configured to transmit respective pressures resulted from the user applying biting force at the first and second cavities. In such cases, relative front-back motion and/or a position of the jaw may cause a characteristic signal from these two cavities to be detected by the processing module 200. In such cases, if the detected motion and/or position of the lower jaw satisfies a criterion, the processing module 200 then generates a feedback signal to cause the feedback signal generator 240 to provide feedback to the user. The feedback is configured to train the user for achieving certain jaw position, and/or grinding activity. Jaw position is implicated in certain disorders including sleep apnea and snoring. By detecting jaw position and/or jaw motion, the apparatus 100 may train the user to maintain a certain jaw position to mitigate one or more of these disorders.

In some cases, the processing module 200 may be configured to detect the position of the tongue of the user. In one implementation, the mouthpiece 300 may be configured to fit at the lower teeth of the user, and a pressure sensor or pressure sensing cavity may be implemented at the mouthpiece 300 behind the front lower teeth. In such cases, gentle pressure by the tongue on the back of the lower teeth may be detected by the pressure sensor or pressure sensing cavity. The processing unit 200 may receive sensed pressure relating to the positioning of the tongue, and determine whether to provide feedback based on a satisfaction of a criterion by the sensed pressure. In some cases, if the sensed pressure is below a threshold, the processing unit 200 may determine that the tongue has moved backward, and may operate the feedback signal generator to provision feedback for the user. For a user suffering from snoring and sleep apnea, the tongue may retract back into the airway, occluding airflow. The feedback is configured to train the user for achieving certain tongue position. In response to the feedback, the user may move his/her tongue forward. By training the user to maintain a specific tongue position (e.g., forward position) during sleep, the apparatus 100 may address snoring and sleep apnea.

In one or more embodiments described herein, the processing module 200 may optionally further include a non-transitory medium. The non-transitory medium may be configured to store a time-series of sensor values (e.g., pressure values, force values, current values, electrical resistance values, etc.) outputted by the pressure sensor 230, a time-series of threshold values (e.g., pressure threshold values, force threshold values, current threshold values, electrical resistance threshold values, etc.) determined by the processing module 200, sound data recorded during a sleeping event of the user, a time at which the feedback is provided to the user by the feedback signal generator 240, or any combination of two or more of the foregoing. The sensor values may be values of the sensor signals provided by the sensor (e.g., pressure sensor 230, force sensor, current sensor, electrical resistance sensor, etc.).

The apparatus 100 described herein may be a part of a system that comprises an electronic device, wherein the apparatus 100 and the electronic device are configured to communicate with each other. In some cases, the electronic device may be configured to: provide a first metric value indicating a level of bite force; allow the user to select whether the user wants the apparatus to provide feedback or not; allow the user to set a feedback threshold value, above which will cause the apparatus to provide the feedback; or a combination of two or more of the foregoing.

By means of non-limiting examples, the electronic device may be a cell phone (e.g., smart watch, Phone), a tablet (e.g., an Pad, miniPad, etc.), a computer, a server, a watch (e.g., iWatch), or any mobile device.

FIG. 6 illustrates a system that includes the apparatus 100 of FIG. 1 and an electronic device 500. In the illustrated example, the electronic device 500 is a smart phone. The apparatus 100 may communicate with the electronic device 500 using a cable or wirelessly. For example, the processing module 200 of the apparatus 100 may include the electrical port 250 for communication with the electronic device 500. Alternatively or additionally, the processing module 200 may include a transceiver configured to wirelessly communicate with the electronic device 500.

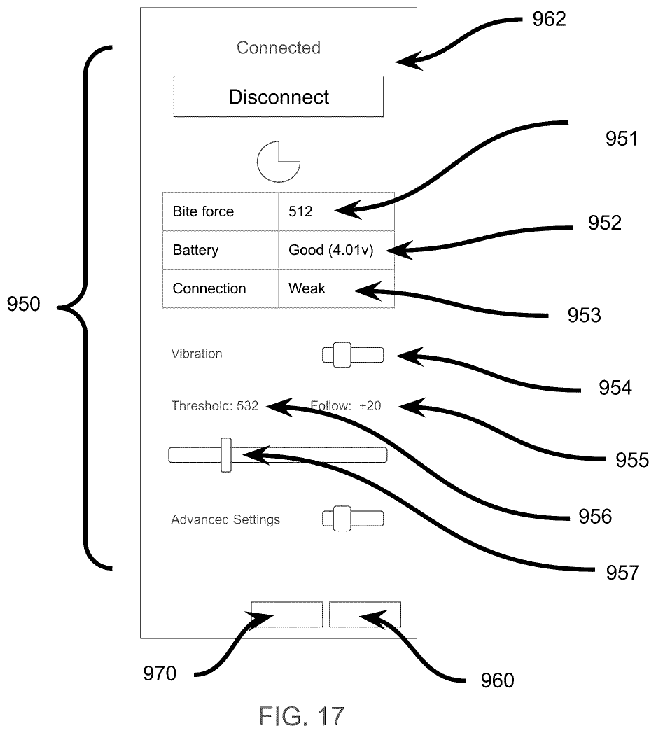

In some embodiments, the electronic device 500 is configured for communication with the apparatus 100 (having a mouthpiece 300). The electronic device 500 includes: a transceiver 960 configured to communicate with the apparatus 100; and a screen 962 configured to display information to a user of the apparatus 100. The electronic device 500 may also include a processing unit 970 configured to process data from the apparatus 100, to process user input relating to operation of the apparatus 100, to determine operation parameter associated with the use of the apparatus 100, or any combination of the foregoing. The electronic device 500 may be configured to: provide a first metric value indicating a level of bite force applied at the mouthpiece 300; allow the user to select whether the user wants the apparatus to provide feedback or not; allow the user to set a feedback threshold value, above which will cause the apparatus 100 to provide the feedback; or a combination of two or more of the foregoing. Features of the electronic device 500 will be further described with reference to FIG. 17.

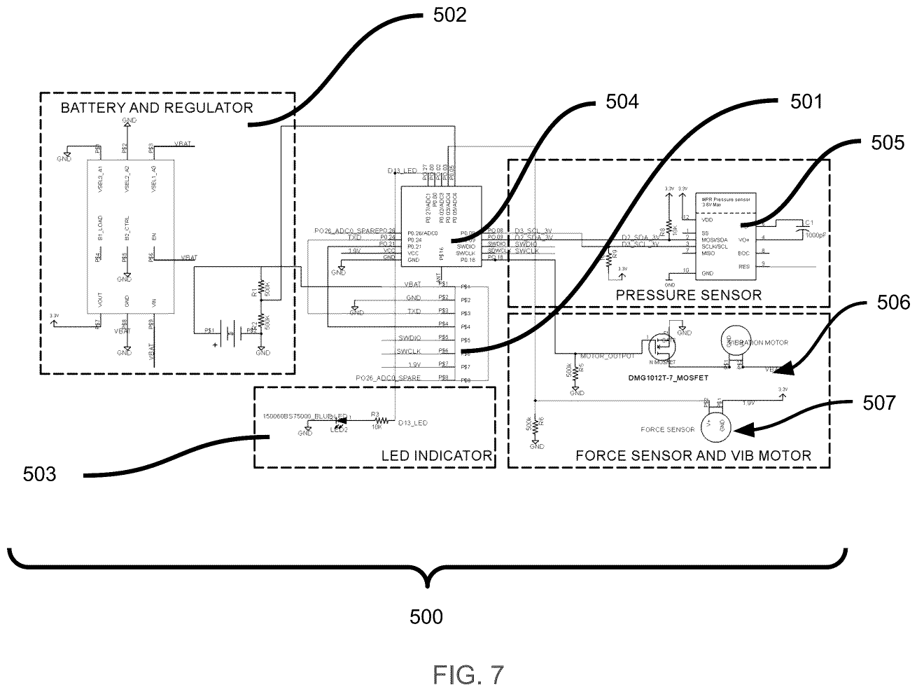

FIG. 7 illustrates an example of a circuit diagram for a circuit 500 in the processing module 200 of FIG. 1. The circuit 500 may be considered as an example of processing unit accommodated by the housing 210 of the processing module 200. As shown in the figure, the circuit 500 includes a connector 501, a power module 502 (which includes a power regulator, and optionally a battery), a LED 503, a processing unit 504, a pressure sensor (with supporting components) 505, a drive circuit 506 for vibration generator (e.g., motor), and a force sensor 507.

The connector 501 is configured for providing power and communication to the processing module 200. The connector 501 may be a USB-C type connector or any of other types of connector. In other embodiments, the connector 501 may be a RF charging circuit to charge the processing module 200 without direct contact with charging pins. This may yield a smaller/lighter device (no connector), and may improve manufacturability (no housing penetrations for a connector).

The power regulator of the power module 502 regulates variable battery power to a known rail voltage for use by the processing module 200. In some embodiments, the power module 502 may include a buck regulator for the power conditioning. Also, in some embodiments, power transduction may be achieved through non-contact power transmission. The LED 503 is configured to provide light to indicate various module states.

The processing unit 504 includes a main processor and Bluetooth radio integrated circuit. The processor is configured to obtain sensor signals from one or more sensors, and output signals to the drive circuit 506 (e.g., for the motor) or LED output devices. The Bluetooth radio of the processing unit 504 transmits data (e.g., user data and/or data associated with an operation of the apparatus 100) to the electronic device 500 (e.g., a phone running an application associated with the apparatus 100).

The drive circuit 506 may be a MOSFET switch controlled by the processing unit 504 to achieve a certain vibration characteristic. The pressure sensor of item 505 may be the pressure sensor 230 of FIG. 1, and the drive circuit 506 may be used to implement the feedback signal generator 240 of FIG. 1. The drive circuit 506, in conjunction with the processing unit 504, may provide various motor vibration profiles to best treat the user. In some cases, the drive circuit 506 may configure a feedback vibration profile specific for the user. This allows the apparatus 100 to effectively titrate up the vibration until a clinical effect is reached, and may have the benefit of not providing higher treatment dose than needed. In some embodiments, the force sensor 507 may be a thin film sensor that can be molded into the mouthpiece 300 to directly measure bite force.

During use, power from the battery is regulated to a usable rail voltage. The rail voltage is applied to the processing unit 504, which obtains sensor values from the pressure sensor 505. The processing unit 504 determines whether the sensor value meets a criterion (e.g., whether the sensor value exceeds a pressure threshold). If the criterion is met, the processing unit 504 then operates the vibration generator to generate feedback for the user. Optionally, the processing unit 504 may also operate the LED 503 to provide visual signal for the user. The visual signal may be feedback to cause the user to alter a behavior. Alternatively or additionally, the LED 503 may provide visual indicator for indicating a status or operating parameter of the apparatus 100, such as power level, charging status, connectivity status, etc. Optionally, the processing unit 504 may also operate a speaker to provide audio signal for the user. The audio signal may be feedback to cause the user to alter a behavior. Alternatively or additionally, the speaker may provide audio indicator for indicating a status or operating parameter of the apparatus 100, such as power level, charging status, connectivity status, etc.

In the above example, the processing unit 504 is configured to determine if a sensor signal (e.g., indicating a sensed pressure) meets a criterion, and if so, the processing unit 504 will generate an operating signal to operate the feedback signal generator (e.g., the vibration generator 506). In some cases, the processing unit 504 may include a comparator for implementing such feature. Also, in some cases, the pressure sensor 230/505 and the feedback signal generator 240 may be implemented as parts of the processing unit 504. For example, the processing unit 504 may include a circuit (e.g., a PCB) implementing the comparator, the pressure sensor 230/505, and the feedback signal generator 240.

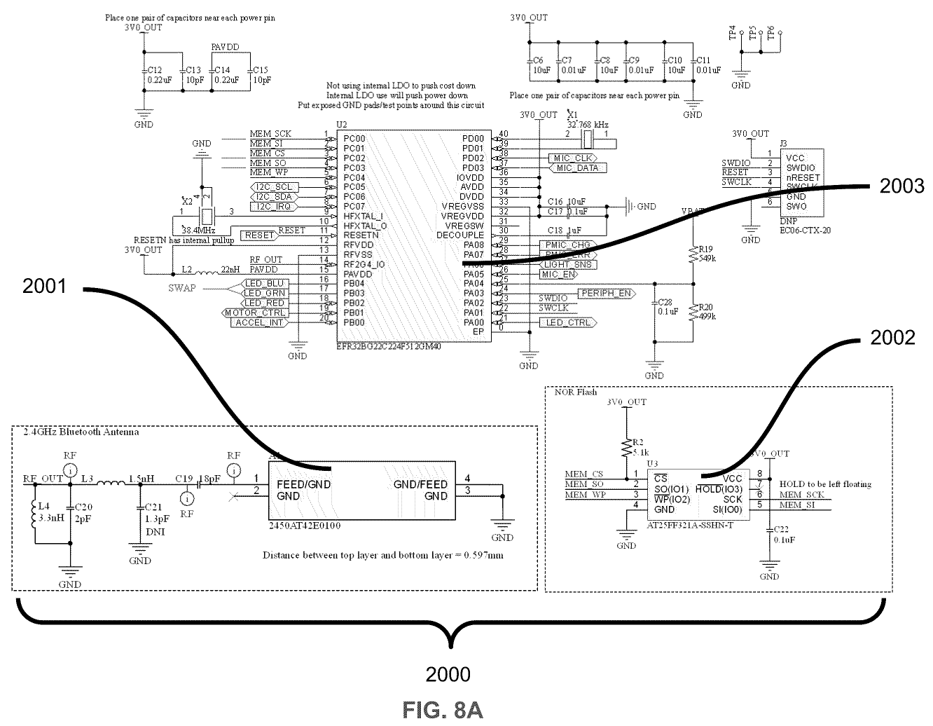

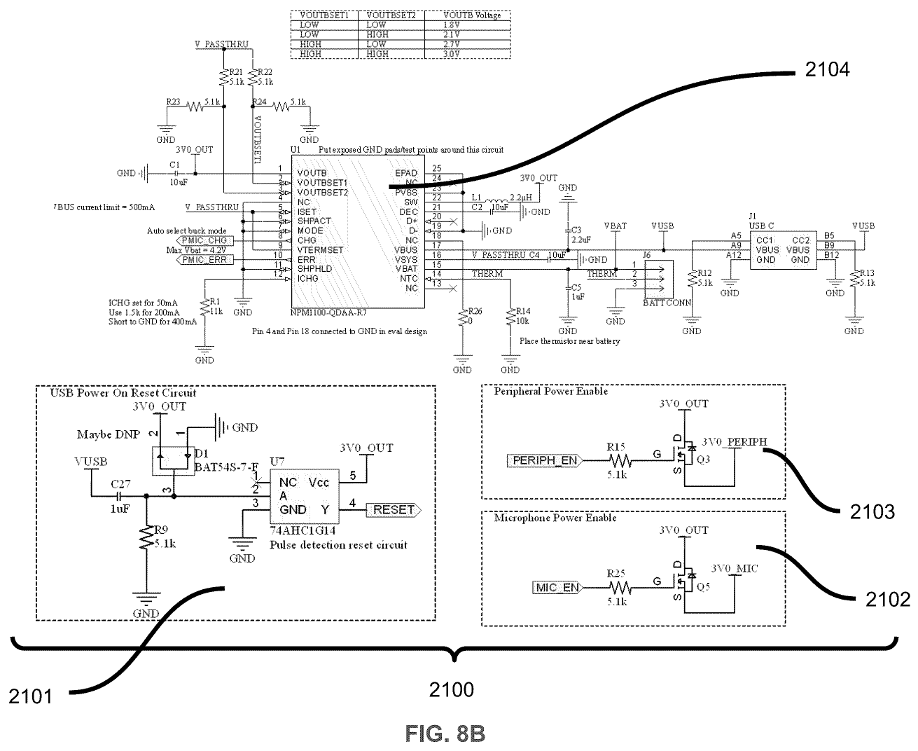

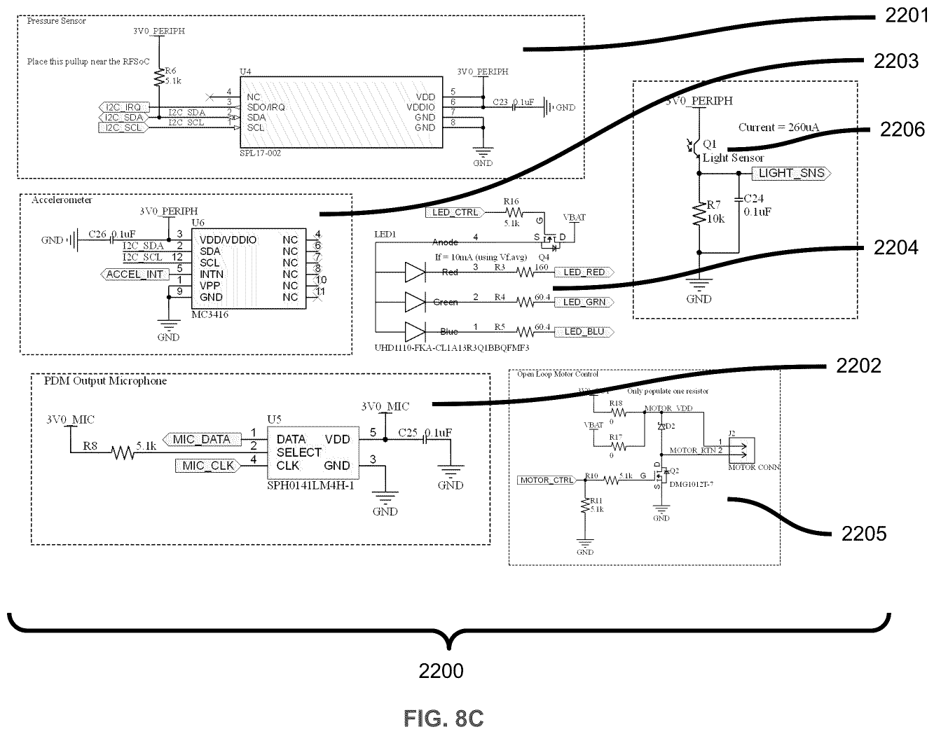

FIGS. 8A-8C illustrate exemplary circuit diagrams of circuits that may be implemented in the processing module 200 of FIG. 1. As shown in the figures, the processing module 200 may include circuits for the Bluetooth radio and processing unit 2000, which includes the Bluetooth antenna circuit 2001 and controller circuit 2003. The circuit 2002 for flash chip to record data is also shown. In some cases, the circuit 2002 may be nonvolatile flash memory implemented in the processing module 200. The nonvolatile flash memory may be used to record data associated with operation of the apparatus 100. For example, nightly user data may be recorded to the flash memory prior to being sync to the electronic device 500 (e.g., phone) and may ultimately be pushed to the cloud. The Bluetooth antenna circuit 2001 is configured to provide a Bluetooth connection to enable data (e.g., user data, data associated with operation of the apparatus 100) to be communicated between from the processing module 200 and the electronic device 500 (e.g., phone). For example, usage data may be transmitted by the processing module 200 to the electronic device 500, and configuration data and setting data may be transmitted by the electronic device 500 to the processing module 200 to configure the apparatus 100.

Alternatively or additionally, the processing module 200 may include power management circuit 2100, which comprises a reset circuit 2101, microphone enable circuit 2102, peripheral power enable circuit 2103, and power management integrated circuit with LiPo cell charger and power (e.g., 3V) conditioning. The power management circuit 2100 is configured to condition the battery power for use in the apparatus 100 (to output a 3.0 volt rail voltage). The circuit 2100 is also configured to charge the LiPo battery and negotiates current draw capabilities over USB for device charging. The reset circuit 2101 power cycles the processing module 200 when USB power is applied to the processing module 200. This permits the apparatus 100 to reset to a known state when plugged in for charging (not in use). Circuit 2102 and circuit 2103 are power enable circuits. The controller circuit 2003 may power down these peripherals when they are not in use to conserve power.

Alternatively or additionally, the processing module 200 may include one or more sensing or feedback component(s) 2200, such as pressure sensor 2201 configured to detect bite pressure, microphone 2202, three-axis accelerometer 2203, LED indicator(s) (e.g., red/green/blue) 2204, a motor drive circuit 2205 that allows the processing unit to create various vibration profiles as feedback to the user, ambient light sensor 2206 configured to detect ambient light, or two or more (e.g., all) of the foregoing. In some cases, there may be multiple pressure sensors 2201 configured to independently obtain pressures representing different degrees of biting applied at different areas of the mouthpiece 300. The microphone 2202 may be configured for detecting snoring and respiration. The accelerometer 2203 may be used as an input and output device. As an output device, the accelerometer 2203 may detect body orientation during sleep. As an input device, the accelerometer 2203 may serve to wake up the processing module 200 from a sleep state by detecting motion.

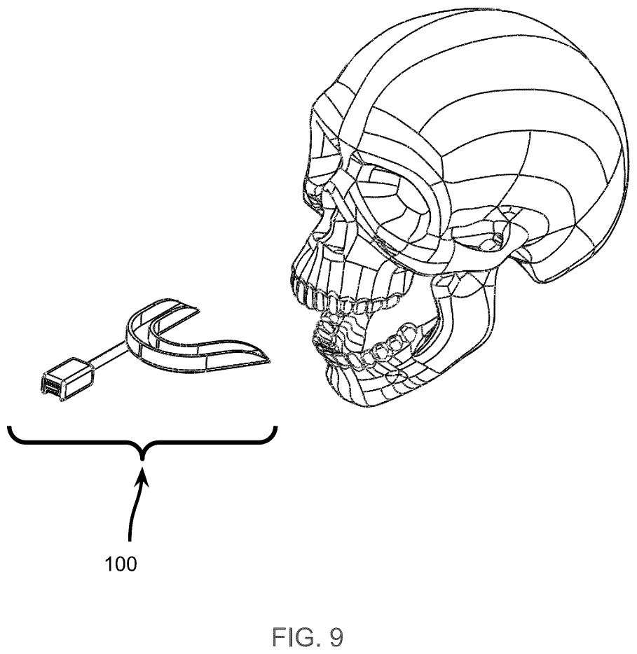

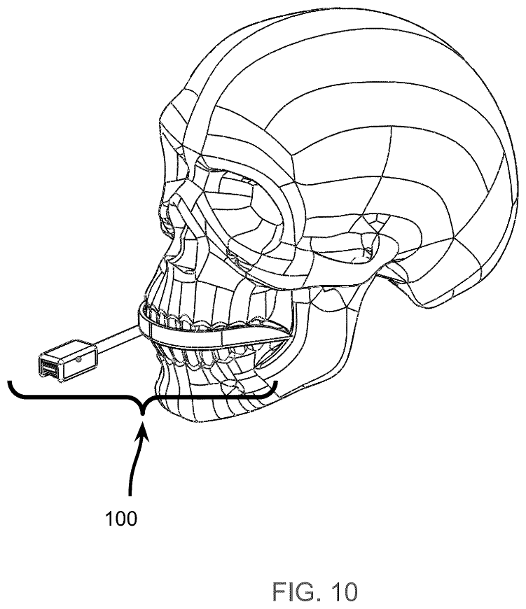

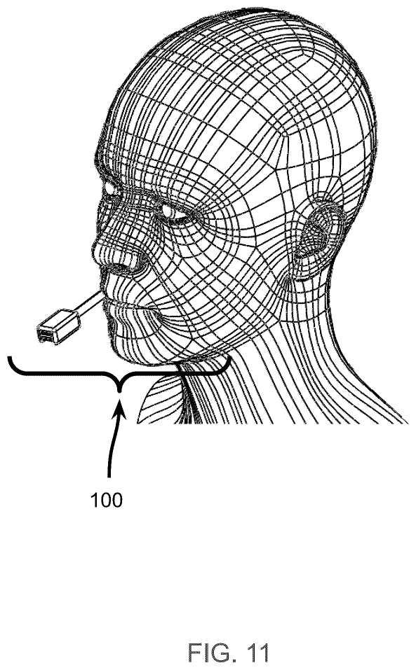

FIGS. 9-11 illustrate a method of using the apparatus 100 of FIG. 1. FIG. 9 illustrates the apparatus 100 of FIG. 1, with the mouthpiece 300 about to be inserted into a mouth of a user. FIG. 10 illustrates the apparatus 100 of FIG. 1, with the mouthpiece 300 being engaged with teeth of the user. FIG. 11 illustrates the apparatus 100 of FIG. 1, with the mouthpiece 300 being inside the mouth of the user.

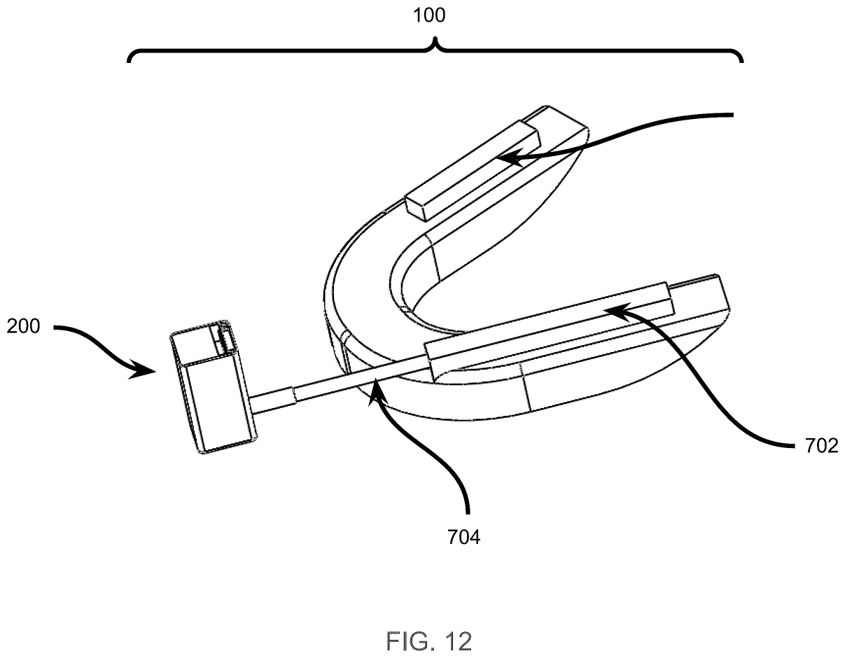

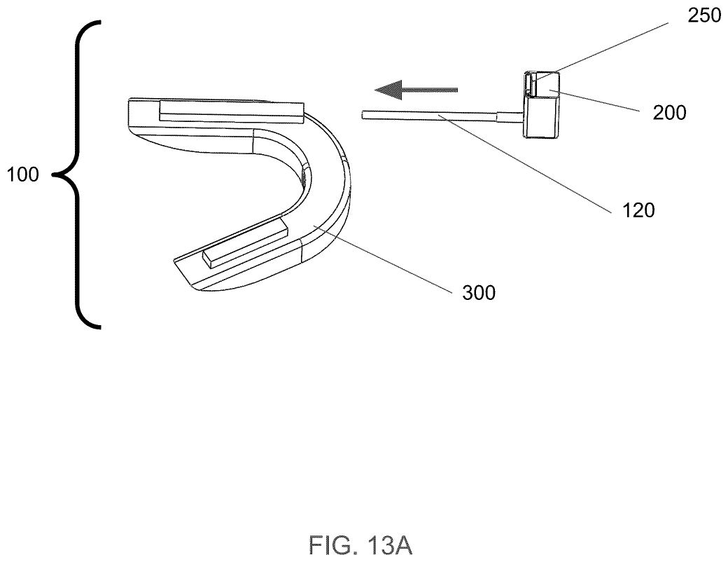

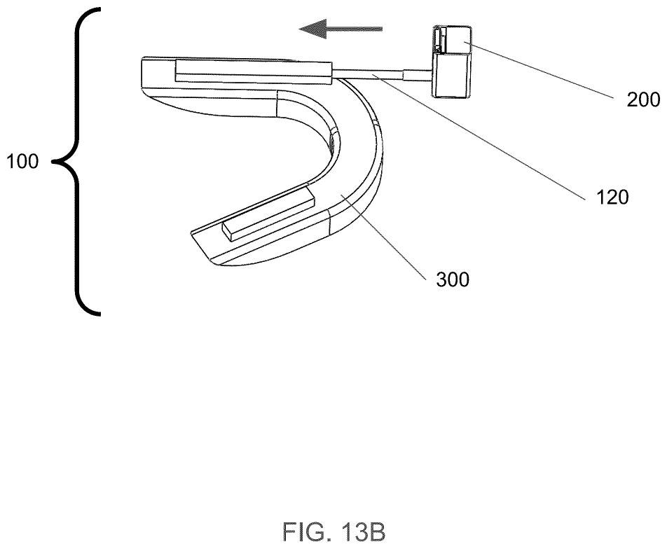

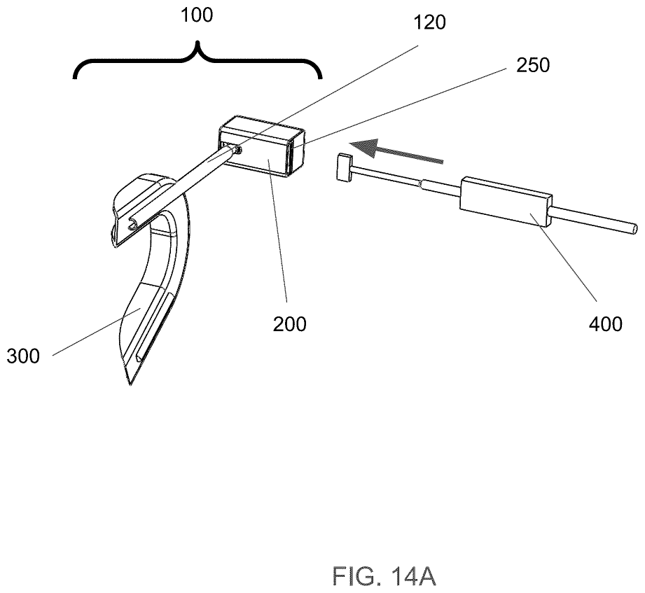

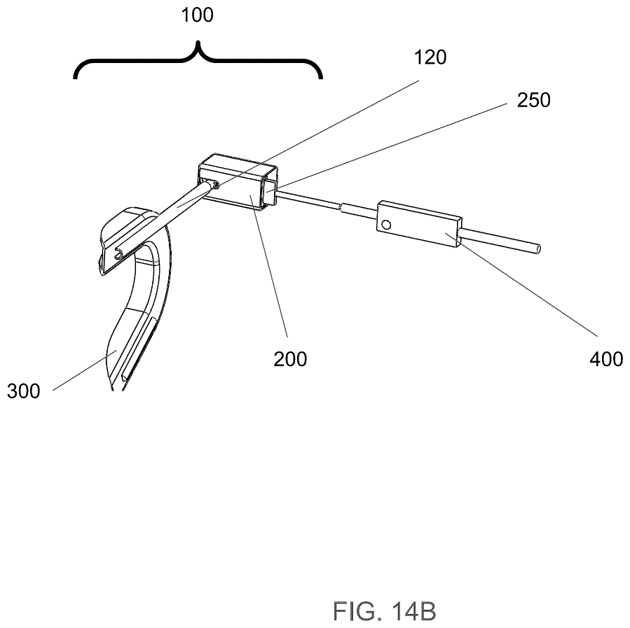

In the above embodiments, the connector 120 is illustrated as extending from a side (e.g., a minor surface) of the processing module 200. In other embodiments, the connector 120 may extend from a major surface of the processing module 200. FIG. 12 illustrates a variation of the apparatus of FIG. 1, particularly showing the tube extending from a long side (e.g., a major surface) of the processing module. As similarly described, the tube 120 may be detachably coupled to the mouthpiece 300. FIG. 13A-FIG. 13B illustrate the tube 120 of the apparatus 100 of FIG. 12 being coupled to the mouthpiece 300 of the apparatus 100. Also, as similarly described, the processing module 200 may include an electrical port 250 to which a charge/communication cable 400 may be connect. FIG. 14A-FIG. 14B illustrate the apparatus 100 of FIG. 12 being coupled to a charge/communication cable 400. In some embodiments, the cable 400 may include visual indicator for indicating a status of the charging. For example, there visual indicator may be implemented using one or more LEDs, which provide different colors for respectively indicating completion of charging, active charging, and a fault of the charging. Alternatively or additionally, the charging status may also be indicated in the screen 962 of the electronic device 500.

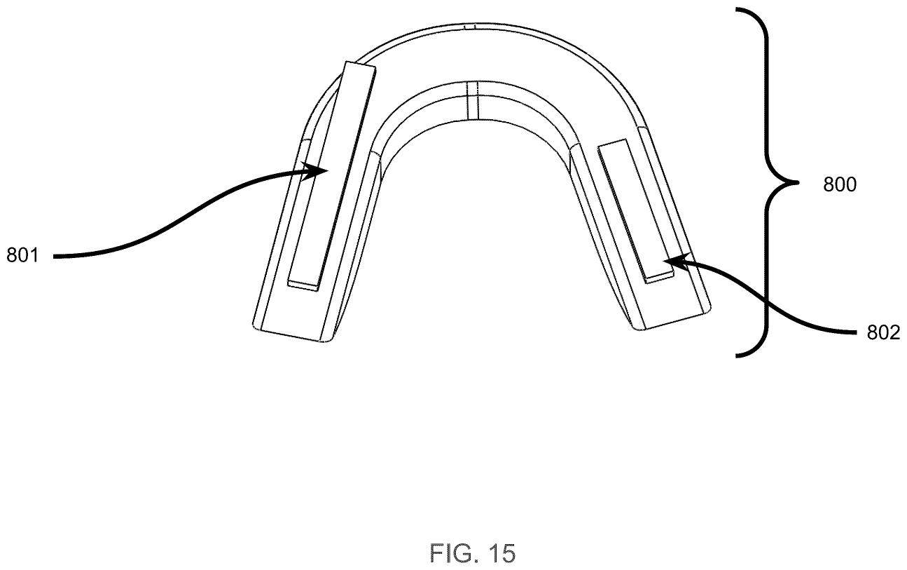

FIG. 15 illustrates another example of the mouthpiece 300 in the apparatus of 100 FIG. 1 or FIG. 12. The mouthpiece 300 of FIG. 15 is similar to that of FIG. 5, except that the mouthpiece 300 of FIG. 15 includes two cavities (first cavity 305a, and second cavity 305b). The cavities 305a, 305b have the same dimension (e.g., cross-sectional dimension). The first and second cavities 305a, 305b are configured to respectively couple to ends of a Y-connector connecting the mouthpiece 300 to the processing module 200. In particular, the connector 120 in such embodiments may be a tube having a Y-configuration. Such Y-connector 120 has a first end configured to couple (e.g., detachably connect) to the first cavity 305a at the mouthpiece 300, a second end configured to couple (e.g., detachably connect) to the processing module 200, and a third end configured to couple (e.g., detachably connect) to the second cavity 305b at the mouthpiece 300. In other cases, the first end and the third end of the Y-connector may be permanently secured to (e.g., integrally formed with) the mouthpiece 300 and are in fluid communication respectively with the first and second cavities 305a, 305b of the mouthpiece 300. During use, the user may apply biting force against the mouthpiece 300, which compresses the first and second cavities 305a, 305b at opposite lateral sides of the mouthpiece 300. The cavities 305a, 305b deform in response to the biting force, and reduce in volume, thereby causing the pressure in the Y-connector to increase. The Y-connector “transfers” the increased pressure to the pressure sensor 230 at the processing module 200, which then detects a bruxing event based on the sensed pressure.

The cavities 305a, 305b may be sandwiched between layers of the mouthpiece 300, or may be implemented using respective tubes that are secured to (e.g., integrally formed with) the exterior surface of the body 303 of the mouthpiece 300. Each tube has a closed first end, and an opposite open end, and is elastically deformable in response to biting force applied by the user. In some embodiments, a first EVA material (or another material) may be molded onto an impression of a user's teeth. Then temporary spacers are placed at the created impression for the cavities 305a, 305b. Then a second EVA material (or another material) may be laminated over the first EVA material, creating the mouthpiece 300. The temporary spacers may then be removed, providing the spaces at the mouthpiece 300 as the cavities 305a, 305b.

In some embodiments, the portion of the mouthpiece 300 surrounding the cavity 305 may have cutouts to permit easy cleaning. In other embodiments, the cutouts are not required, and the mouthpiece 300 does not include the cutouts.

In some cases, instead of using both cavities 305a, 305b to connect the mouthpiece 300 to the processing module 200, only one of the two cavities 305a, 305b may be used (like that described with reference to FIG. 5). In such cases, the tube (connector) 120 may have only two opposite tube ends (i.e., no Y-configuration) for unilateral bite detection, and the mouthpiece 300 may further comprise a spacer configured for placement in either the first cavity 305a or the second cavity 305b. For example, if the first cavity 305a is used to couple to the first end 122 of the tube (connector) 120, then the spacer may be inserted into second cavity 305b, and vice versa. The spacer is advantageous because it provides an even support for the mouthpiece 300 so that when the user applies biting force, the left and right teeth will engage with the left and right sides of the mouthpiece 300, thereby balancing the bite. Providing bilateral cavities 305a, 305b is also advantageous because it allows the user to select the preferred side for the pressure sensing tube 120.

In some embodiments, spacers with different respective thicknesses may be provided. Each spacer may be selectively inserted to, and removed from, one of the cavities 305. This allows the user to apply different spacers of varying thicknesses to determine which one provides the best comfort.

Also, in some embodiments, a spacer, a linear tube, and a Y-tube may be provided with the apparatus 100. This allows the user to physically configure the apparatus 100 in different ways to achieve different uses. For example, the user may couple the linear tube to a left side of the mouthpiece 300, and insert the spacer to the right cavity 305. The user may alternatively couple the linear tube to a right side of the mouthpiece 300, and insert the spacer to the left cavity 305. The user may also alternatively couple the Y-tube to the left and right sides of the mouthpiece 300, in which case, the linear tube will not be used.

In some cases, the processing module 200 may include two sensors 230 configured to respectively coupled to the right and left cavities 305a, 305b of the mouthpiece 300 via respective tubes (connectors) 120. This feature is advantageous because it allows detection of biting at the left and right sides independently (bilateral biting detection). As described herein, the processing module 200 may utilize such data to determine whether the user is grinding his/her teeth.

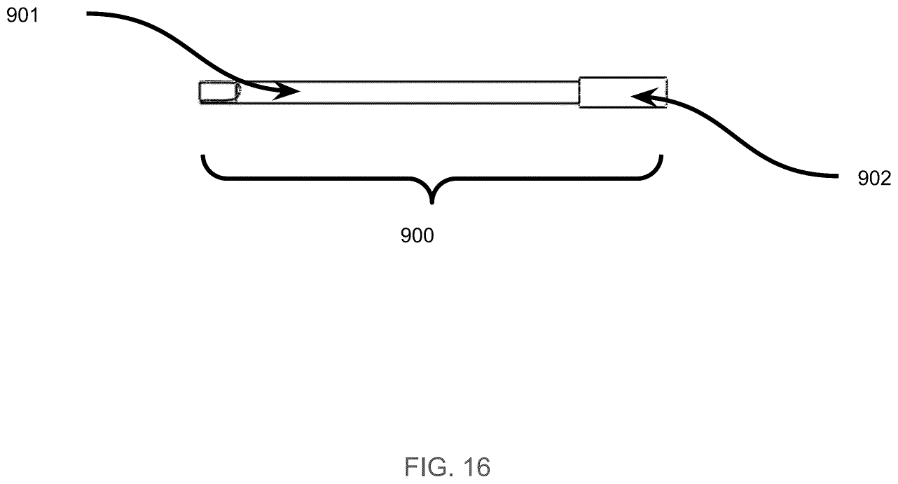

FIG. 16 illustrates an example of connector 120 in the apparatus 100 of FIG. 1 or FIG. 12. The connector 120 is a tube having a first end 122 configured to couple to the mouthpiece 300, a second end 124 configured to couple to the processing module 200, and a tube body 901 extending between the first end 122 and the second end 124. In some embodiments, the tube body 901 has a rigidity that is sufficiently low so the tube body can be elastically deformed to reduce a volume inside the lumen of the tube body 901 in response to biting force exerted by the user. The tube body 901 should also have a rigidity that is sufficient high (e.g., not too compliant) so that the tube body 901 can transmit vibrational force as feedback from the processing module 200 to the user via the mouthpiece 300. The second end 124 of the tube body 901 is coupled to the pressure sensor 230 via a sealing tube 902. The sealing tube 902 may be made from silicone, plastic, a polymer, or any of other suitable materials. In one implementation, the sealing tube 902 may be a heat-shrink tube disposed over the second end 124 of the tube (connector 120). In another implementation, the tube body 901 and the sealing tube 902 may be created by overmolding. In such cases, the tube body 901 and the sealing tube 902 form a unity configuration, and are permanently fixed to each other.

In some cases, the tube body 901 may be made from a rigid plastic. In other cases, the tube body 901 may be made from an elastomer, such as silicone. The tube body 901 may be made from other materials in other embodiments.

FIG. 17 illustrates an example of the electronic device 500 configured to communicate with the apparatus 100 of FIG. 1 or FIG. 12. The electronic device 500 includes a transceiver 960 configured to communicate with the apparatus 100, and a screen 962 configured to display information to a user of the apparatus 100. The electronic device 500 also includes a processing unit 970 configured to perform one or more functions, and/or to assist in implementing one or more features, described herein.

In the illustrated embodiments, the electronic device 500 is configured to provide a first metric value 951 indicating a level of bite force applied at the mouthpiece. In some cases, such may be accomplished by the electronic device 500 establishing a connection (wired or wireless connection) with the apparatus 100, and receiving the first metric value from the apparatus 100. During use, the processing module 200 of the apparatus 100 may monitor a level of biting force exerted by the user against the mouthpiece 300. If a connection is established between the electronic device 500 and the apparatus 100, the electronic device 500 may receive the biting force (or other parameter indicating force or severity of the biting) from the apparatus 100. The electronic device 500 may then display such information as the first metric value 951 in the screen 962. In the illustrated example, biting force value is displayed by the electronic device 500. In other embodiments, the electronic device 500 may be configured to display other metrics indicating the level of the biting force. For example, the electronic device 500 may display pressure value, current value, electrical resistance value, etc., or any of other values that may indicate a severity of the biting performed by the user.

As shown in FIG. 17, the electronic device 500 also provides a battery level indicator 952 indicating a level of battery power in the apparatus 100. The battery power level may be transmitted by the apparatus 100 to the electronic device 500, which then displays battery power information as the battery level indicator 952 at the screen 962 of the electronic device 500. The battery power information may be the battery power level, or information (e.g., battery status indicator, such as “Excellent”, “Good”, “Low”, etc.) derived from the battery power level.

The electronic device 500 also provides a connection level indicator 953 indicating a level of the connection strength between the electronic device 500 and the apparatus 100. The connection strength level may be transmitted by the apparatus 100 to the electronic device 500, which then displays connection level information as the connection level indicator 953 at the screen 962 of the electronic device 500. The connection level information may be the connection strength level, or information (e.g., “weak”, “good”, “strong”, etc.) derived from the connection strength level.

The electronic device 500 also provides a mode control 954 configured to allow a user of the apparatus 100 to select an operation mode for the apparatus 100. For example, the user can use the mode control 954 to turn off vibration (e.g., placing the apparatus 100 in a monitoring mode without the need to receive vibrational feedback from the apparatus 100), or to turn on vibration. When the apparatus 100 is operating in the vibration mode, the apparatus 100 provides feedback to the user in response to detected bruxing event. In one implementation, after the user has operated the mode control 954 at the electronic device 500, the electronic device 500 then transmits a corresponding mode control signal (e.g., via a cable or wirelessly) to the apparatus 100. Upon receiving the mode control signal, the processing module 200 of the apparatus 100 is then configured by the mode control signal to either provide monitoring function only, or to provide feedback for the user. The processing module 200 may include a non-transitory medium storing mode control data indicating the current mode of the apparatus 100.

Optionally, the electronic device 500 may provide other mode(s) for selection by the user. For example, in some cases, the electronic device 500 may provide a mode control for allowing the user to select whether to monitor snoring or to provide feedback to address snoring.

Also, in some cases in which the apparatus 100 includes other feedback signal generator(s), such as speaker and/or LED, the electronic device 500 may provide corresponding feedback control(s) for allowing the user to select whether to receive vibration feedback, audio feedback, visual feedback, or any combination of two or more of the foregoing, to address a medical condition, such as bruxing, snoring, etc. After the feedback control(s) has been set by the user, the electronic device 500 then transmits a corresponding feedback control signal (e.g., via a cable or wirelessly) to the apparatus 100. Upon receiving the feedback control signal, the processing module 200 of the apparatus 100 is then configured by the feedback control signal to operate the vibration force generator to provide vibration feedback, to operate the speaker to provide audio feedback, to operate a LED to provide visual feedback, or any combination of two or more of the foregoing.

The electronic device 500 may also provide a user control 957 (a slider in the illustrated example) for allowing the user to set a threshold 956 for providing feedback. After the threshold 956 is set, the electronic device 500 may then communicate such threshold 956 to the apparatus 100. Upon receiving the threshold, the processing module 200 of the apparatus 100 then use the threshold 956 for determining whether to provide feedback to the user of the apparatus 100. During use of the apparatus 100, if the processing module 200 of the apparatus 100 determines that the biting force, or a metric representing a severity of biting, is above the threshold 956, the processing module 200 then operates the feedback signal generator 240 to provide feedback for the user.

In some cases, the information displayed on the screen 962 as shown in FIG. 17 may be provided by the electronic device 500 while the electronic device 500 is communicatively connected to the apparatus 100, and while the user is wearing the mouthpiece 300. For example, the first metric value 951 (indicating a level of the biting) being displayed at the screen 962 of the electronic device 500 may be real time information obtained while the user is applying biting force against the mouthpiece 300. The electronic device 500 also displays a difference value 955 that is calculated as a difference between the first metric value 951 and the threshold 956. Thus, in one method of use, the user may use the interface of the electronic device 500 to determine an estimate of a baseline biting force by gently closing his/her mouth, and reading the first metric value 952. The user may then set the threshold 956 to be a certain value (difference value 955) over the estimated baseline biting force. The threshold 956 may be transmitted to the apparatus 100 for determining when to apply feedback to the user. In some embodiments, the threshold 956 may be a fixed threshold. In other embodiments, the threshold 956 may be utilized as an initial threshold, and the apparatus 100 may further adjust the threshold during use, as described herein.

In some cases, at the beginning of use, the electronic device 500 (e.g., an application therein) may prompt the user to go through a bite-release protocol. The electronic device 500 may then determine the threshold as a percent of the pressure difference achieved during this protocol. The electronic device 500 may prompt a bite-release protocol to determine what a “bite” looks like for a particular user. For example, the user may be asked by the electronic device 500 to proceed through a sequence of steps at the start of treatment such as bite-then-release. This sequence can be used to clarify a bite and a non-bite pressure. The threshold may then be set by the electronic device 500 as a non-bite (baseline) pressure plus a percentage of pressure difference between non-bite (baseline) pressure and bite pressure. For example, if during this calibration sequence, a bite is determined to be 200 and non-bite is determined to be 100, and if the percentage is 25%, the threshold may be determined and set as (200-100)×1.25=125. In other cases, the threshold may be set by the electronic device 500 as a factor times the non-bite (baseline) pressure. For example, if the non-bite (baseline) pressure is 120, and if the factor is 1.2, then the threshold may be determined and set as 120×1.2=144.

The bite-release protocol is advantageous because it accounts for bite force and bite mechanics that are user-specific, and how the user wears the mouthpiece and interacts with the tube 120. Furthermore, the bite-release protocol serves as a self-test of the apparatus 100 to ensure everything is working (e.g., no pressure leak in the system).

Also, in some cases, the electronic device 500 may obtain and store data logged by the apparatus 100. While the user is using the apparatus 100, the apparatus 100 may obtain and store various information associated with the operation and use of the apparatus 100. For example, the apparatus 100 may obtain and store time series of metric data indicating a level of biting force over time, time points at which feedback was provided to the user, sound data indicative of snoring and/or breathing, time points at which bruxing events were detected, time points at which sleep apnea events were detected, etc. When the apparatus 100 is communicatively connected to the electronic device 500, the stored data in the apparatus 100 may then be downloaded to the electronic device 500.

It should be noted that one or more of the features of the electronic device 500 described herein may be implemented using hardware (e.g., processor(s), circuit component(s), integrated circuit, etc.), software (e.g., coding, script, application, etc.), or a combination of both. Also, the electronic device 500 may be a cell phone (e.g., a smart phone, Phone), an Pad, a tablet, a laptop, a computer, a smart watch, or any of other types of communication devices operated by the user of the apparatus 100. In one implementation, the electronic device 500 is a cell phone, and the user of the cell phone may download an application provided by the provider (e.g., manufacturer, seller, etc.) of the apparatus 100. The application is configured to provide one or more features described with reference to FIG. 17.

In some embodiments, the electronic device 500 may be configured to provide one or more other features. For example, the electronic device 500 (e.g., the application in the electronic device 500) may remind the user to begin using the apparatus 100, may inform the user that he/she no longer need to use the apparatus 100 if the bruxing has subsided, may present a bruxism trend to user showing the progress being made by the user to address the bruxing, may present patient progress towards specific goals, may track and/or display a duration and/or a number of bruxing events per night, may track and/or compare bruxing activity between a patient and activities of his/her friends, may perform a self-test, may perform a calibration of one or more sensors at the processing module 200, or any combination of two or more (e.g., all) of the foregoing.

Although exemplary features of the apparatus 100 are described, it should be noted that the apparatus 100 is not limited to having such features. In other embodiments, the apparatus 100 may include only one, or a subset of the features described herein. In other embodiments, the apparatus 100 may include one or more additional features. In further embodiments, the apparatus 100 may include all of the features described herein.

In some embodiments, the apparatus 100 may serve as a training tool to help user manage and develop better habits with regards to nighttime behavior (e.g., teeth grinding, jaw position, breathing, etc.). The apparatus 100 may provide feedback to the user to treat nighttime ailments, such as bruxing, snoring, sleep apnea, etc.

In the embodiments in which the apparatus 100 includes one or more microphones, the microphones of the apparatus 100 may be positioned in front of the lips of the user to detect snoring and apnea events. For example, microphones are configured to detect sound while the user is sleeping, and the processing module 200 is configured to evaluate and categorize sounds as either breathing or snoring. In some cases, the processing module 200 may determine that there is an apnea episode based on lack of breathing sound, or based on sound of struggling breathing. If snoring and/or apnea is detected, the processing module 200 then operates one or more feedback signal generators 240 (e.g., vibrational force generator) to provide feedback to the user. The feedback may have an intensity sufficient to cause the user to stop the snoring and/or the apnea episode. In some embodiments, the processing module 200 may also determine sleeping parameters to determine a quality of sleep. For example, the processing module 200 may process microphone signals from the microphone(s) to determine snoring frequency, snoring amplitude, breathing rate, breathing period etc., or any combination of two or more of the foregoing. The processing module 200 may also detect sleeplessness based on one or more of these parameters. In the embodiments in which the apparatus 100 includes accelerometer, the processing module 200 may utilize output from the accelerometer to determine sleeplessness (e.g., the accelerometer output may indicate that the user is turning and tossing frequently). Thus, the apparatus 100 may be utilized to monitor, track and/or regular activities during sleep.

In some embodiments, the apparatus 100 may optionally include a pulse oximeter. The pulse oximeter may be implemented at the mouthpiece 300 or at the tube (connector) 120. In some cases in which the processing module 200 is integrated into the mouthpiece 300, the pulse oximeter may be implemented in the processing module 200. The pulse oximeter is configured to obtain oxygen saturation levels in the mouth, which may be more accurate than those measured on the digits. Oxygen saturation levels obtain from the pulse oximeter may be used by the apparatus 100 to determine if a user is experiencing sleep apnea. If the processing module 200 determines that the pulse saturation level is below a certain level (and optionally, longer for a certain time duration), the processing module 200 may then signal the feedback signal generator 240 to provide feedback to the user. The feedback may be at an intensity sufficient to cause the user to stop the apnea episode.

In some embodiments, the apparatus 100 (optionally together with the electronic device 500) may be configured to provide certain training for a specific user. Such training may be provided over time with the goal of eliminating or reducing certain adverse conditions, such as bruxing, snoring apnea, etc. In some cases, a fixed training program may be provided for different users. In other cases, the training program may be individually tailored for each user. In some embodiment, a program or AI agent may use user profile data, and data obtained by the apparatus 100 to adjust characteristic of the apparatus 100 to improve or optimize training. For example, the program/AI agent may assess the bite force data, and automatically sets the vibration thresholds to most quickly achieve training. In some embodiments, the program/AI agent may obtain data of multiple users of the apparatuses 100, and determine the best threshold for feedback provision and feedback strength for a specific user. In some embodiments, the program/AI agent may remind the user to use the apparatus 100 to reinforce the learning previously achieved by using the apparatus 100. For example, an alert may be sent to the user once a month to use the apparatus 100 to confirm that bruxing level, snoring level, or apnea level is at an acceptable level. The program/AI agent may be implemented in the apparatus 100, in the electronic device 500, or a combination of both.

In some cases, the apparatus 100 and/or the electronic device 500 may be configured to automatically reduce the threshold for providing feedback to the user. Over the course of the training period, the apparatus 100 and/or the electronic device 500 may gradually lower the threshold as the user learns the proper behavior (e.g., not to grind his/her teeth). For example, on night #1, the threshold may be set at level 200. The apparatus 100 and/or the electronic device 500 may assess the user's performance to determine whether the threshold can be reduced. For example, if the user's bite force level and/or frequency of biting has reduced to acceptable level(s), the apparatus 100 and/or the electronic device 500 may then determine that the threshold for providing feedback can be reduced. The threshold may then be set to 180 on night #2, for example. In this way, the system considers the performance of the user and gradually reduce threshold for providing feedback in order to achieve less and less bite force.

In some embodiments, the apparatus 100 and/or the electronic device 500 may select and/or adjust thresholds for feedback provision, varying vibration (or other) feedback amplitudes, varying timing of stimuli, adjust parameters based on age, or sex, or other user-specific traits. In some embodiments, the feedback amplitude (e.g., vibration amplitude, sound amplitude, light amplitude, temperature amplitude, electrical discharge amplitude, air force amplitude, etc.) may be adjusted (e.g., dynamically during sleep, or before or after each use). In some embodiments, the time delay between bite and feedback may be adjusted (e.g., dynamically during sleep, or before or after each use). In some embodiments, the required number of nights for using the apparatus 100 may be adjusted (e.g., dynamically during sleep, or before or after each use). In some embodiments, the number of nights between use of the apparatus 100 may be adjusted (e.g., dynamically during sleep, or before or after each use).

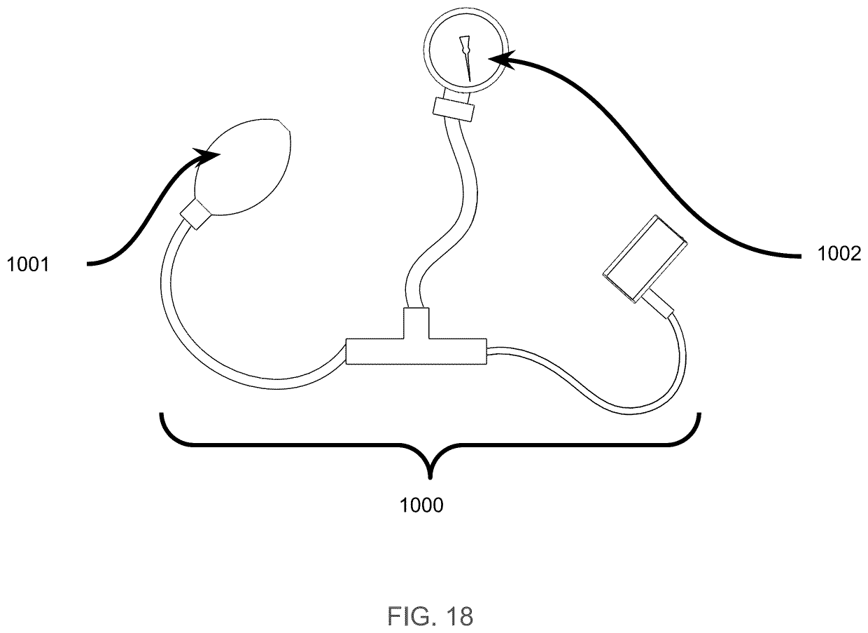

FIG. 18 illustrates a pressure testing apparatus 1000 configured to obtain pressure reading of the apparatus 100 of FIG. 1 or FIG. 12. The pressure testing apparatus 1000 includes a squeezable bulb 1001, a gauge 1002, and tubing 1004 connecting the bulb 1001, the gauge 1002, and the apparatus 100 to each other. During the pressure testing, the bulb 1001 is squeezed to pressurized the tubing 1004, which simulates a biting action. The gauge 1002 indicates the pressure within the tubing 1004. The pressure is also sensed by the pressure sensor 230 of the apparatus 100. The sensed pressure by the apparatus 100 is compared with the gauge reading to determine if they match or if they are within a threshold from each other. If so, then the apparatus 100 may be considered as passing the pressure testing.

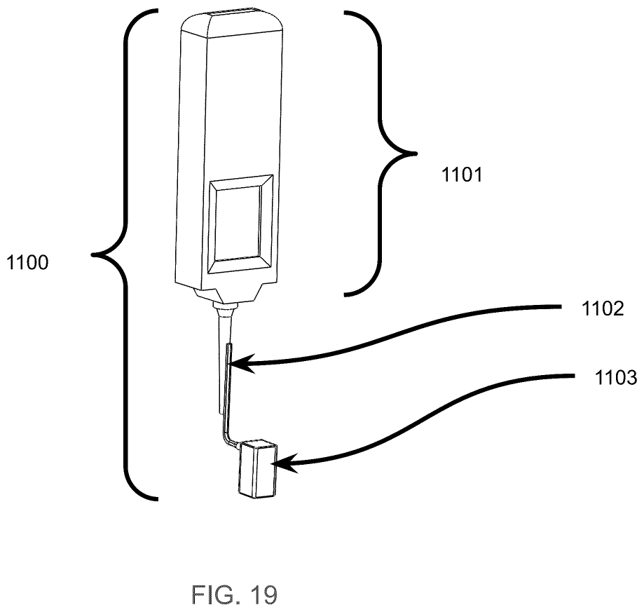

FIG. 19 illustrates a vibration testing apparatus 1100 configured to test a vibrational force provided by the apparatus 100 of FIG. 1 or FIG. 12. The vibration testing apparatus 1100 includes vibrometer 1101, and an adapter 1102. During testing, the adapter 1102 is coupled between the processing module 200 being tested and the vibrometer 1101. The processing module 200 is being tested by the vibrometer 1101 for vibration. In particular, vibration output from the processing module 200 is tested to confirm that the processing module 200 is able to deliver a peak acceleration of 0.5-15 m/s\hat{ }2 in this setup. This permits the apparatus 100 to deliver a low stimulus to the patient, and ramp up gradually to adjust the vibration until a desired effect (a relaxing of the jaw) is obtained.

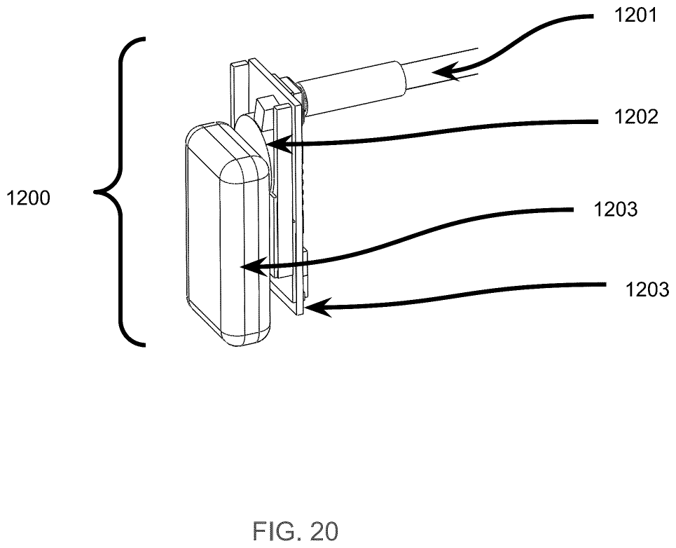

FIG. 20 illustrates exemplary components in the processing module 200 of the apparatus 100 of FIG. 12. The processing module 200 includes a pressure tube connector 1201, a feedback signal generator 240 (e.g., a vibration force generator), a printed circuit board (PCB) 1203, a pressure sensor 230, and a battery 223. In the illustrated example, the pressure sensor 230 and the feedback signal generator 240 are coupled to the PCB 1203. The pressure sensor 230 and/or the feedback signal generator 240 may be considered to be parts of the PCB 1203 in some cases. The pressure sensor 230, the feedback signal generator 240, and the PCB 1203 may be considered parts of a processing unit in the processing module 200. The pressure tube connector 1201 interfaces between the connector 120 of the apparatus 100 and the pressure sensor 230.

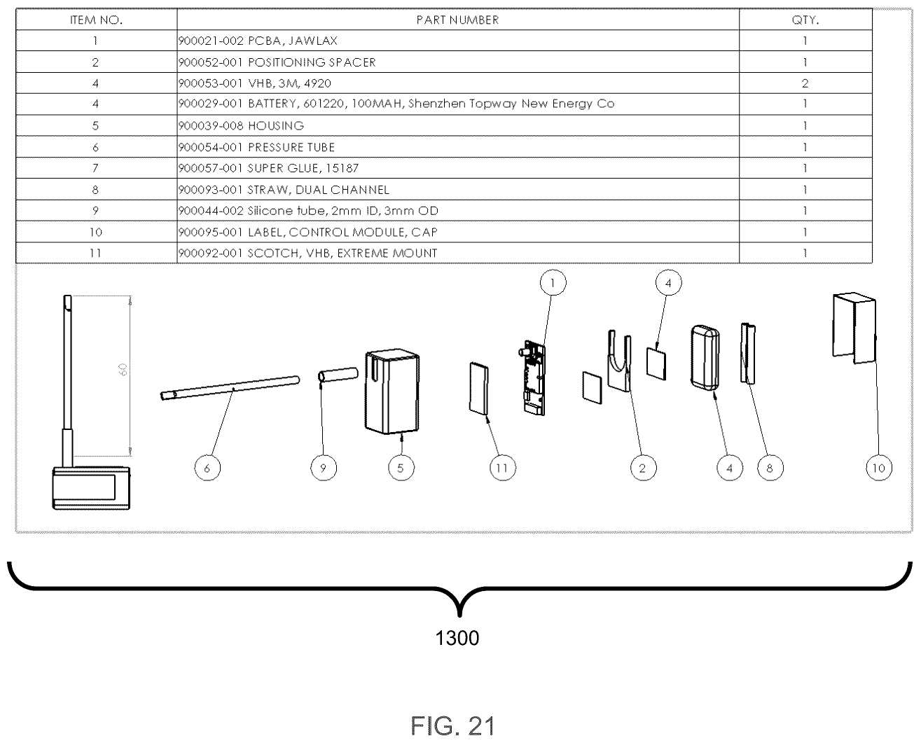

FIG. 21 illustrates exemplary components of the apparatus 100 of FIG. 12. The apparatus 100 includes a processing unit 1302, a spacer 1304, a double sided adhesive 1306, a battery 1308, a housing 1310, a pressure tube 1312 (for implementing the connector 120), a heat shrink tube 1314, a control unit label 1316, and a backer tube 1320. In the illustrated example, the processing unit 1302 is a PCB that implements

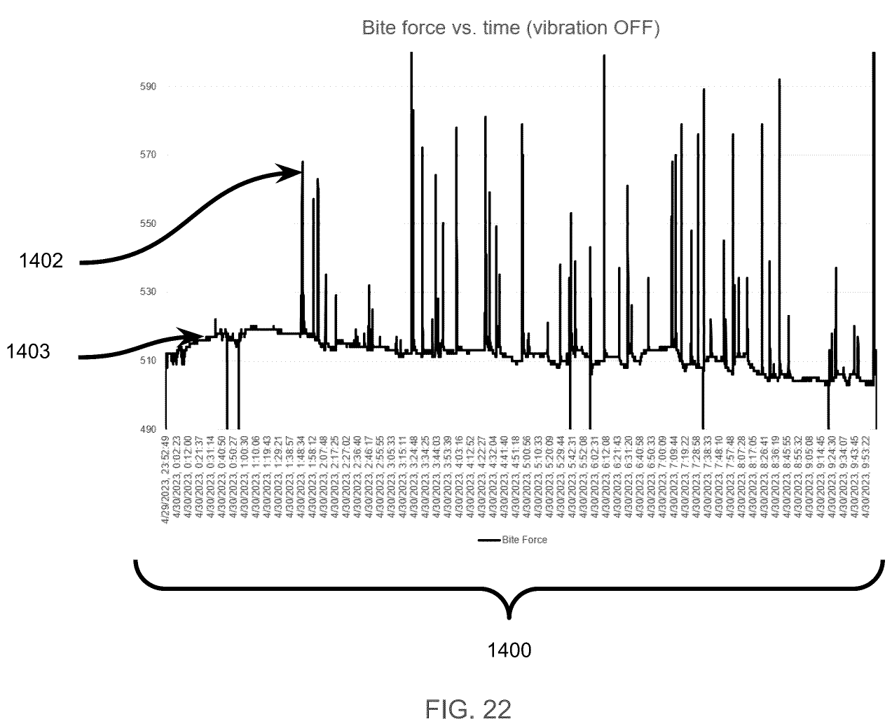

FIG. 22 illustrates example of data captured by the apparatus of FIG. 1 or FIG. 12, particularly showing the data having metrics indicating levels of bite force over time. As shown in the figure, the x-axis of the plot indicates time, and the y-axis indicates levels of biting force. The data in the plot is generated when the apparatus 100 is in a monitoring mode. In the monitoring mode, the feedback signal generator 240 is turned off so that the apparatus 100 does not provide feedback to the user. As shown in the plot, the biting force during a bruxing event 1402 has a significantly higher value (spikes) compared to non-bruxing event 1403. Thus, the apparatus 100 may be utilized to capture data that quantitatively measures a level of teeth grinding. This is advantageous over the qualitative technique in which a dentist diagnoses bruxing by observing wear at the teeth surfaces.

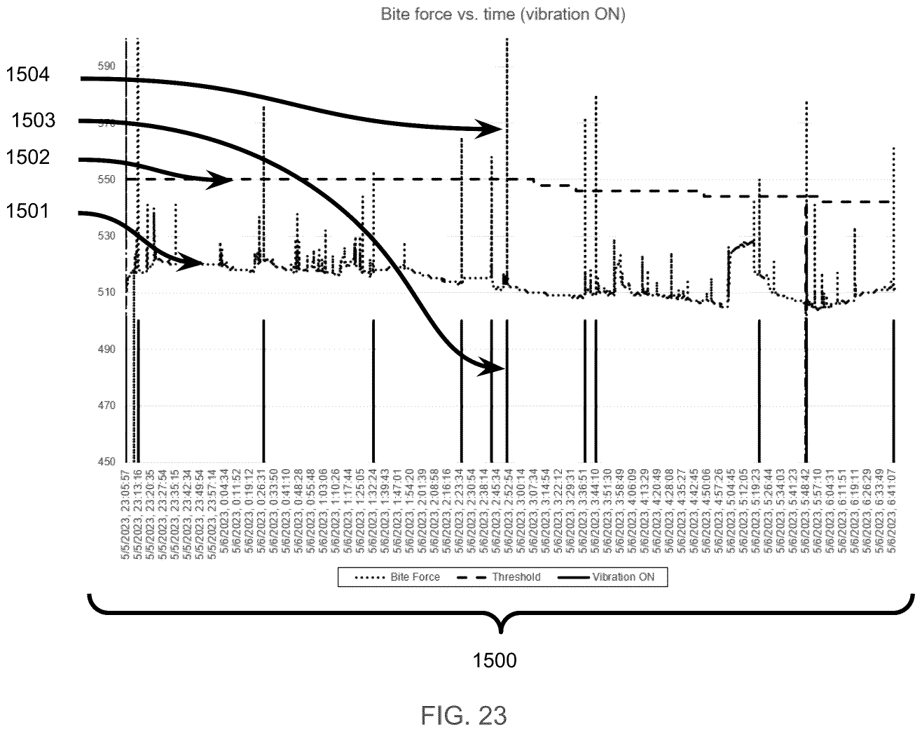

FIG. 23 illustrates example of data captured by the apparatus of FIG. 1 or FIG. 12, particularly showing the data having metrics indicating levels of bite force over time, threshold values for triggering feedback, and timepoints at which feedback was provided. As shown in the figure, the x-axis of the plot indicates time, and the y-axis indicates levels of biting force. The data in the plot is generated when the apparatus 100 is in a feedback mode. In the feedback mode, the feedback signal generator 240 is turned on so that the apparatus 100 provides feedback to the user in response to a detected bruxing event. The feedback may cause the user to stop the bruxing event, and may be utilized to train the user over time so that the user can eventually stop bruxing or reduce the frequency of bruxing even without the use of the apparatus 100.

As shown in FIG. 23, data 1501 are biting force values captured by the apparatus 100 during use (e.g., while the user is sleeping). Data 1502 are threshold values determined by the processing module 200 of the apparatus 100. The processing module 200 is configured to determine a reference threshold based on the threshold values, and to compare the captured biting force with the reference threshold. In one implementation, the reference threshold is a variable reference threshold that is determined by the processing module 200 based on an average of biting forces within a moving temporal window (e.g., the last 10 seconds, 20 seconds, 30 seconds, 1 minute, 2 minutes, 3 minutes, 4 minutes, 5 minutes, etc.). In the illustrated example shown in the figure, the reference threshold is determined by the processing module 200 as a factor F times the calculated average biting force, wherein the factor F is 1.2. The factor F may have other values in other embodiments. In other cases, the calculated average biting force itself may be set as the reference threshold. When calculating the average of biting forces, the biting force associated with bruxing event is excluded from the calculation. This way, the average biting force determined by the processing module 200 will represent a baseline biting force level for non-bruxing event. In other embodiments, instead of the variable reference threshold, the reference threshold may be a constant value set by the user, or automatically by the processing module 200. Data 1503 indicates provision of feedback for the user. It can be seen that whenever the biting force 1501 exceeds the threshold 1502, the processing module 200 then determines that there is a bruxing event (indicated by spikes 1504), and the apparatus 100 then provides feedback to the user. Immediately after the feedback is provided to the user, the bruxing event stops for a duration.

Using the variable reference threshold to detect bruxing events is advantageous because the user's baseline biting force (for non-bruxing event) may change during sleep. For example, the user may tend to relax the jaw more as the night progress. The variable reference threshold accounts for this variable baseline biting force so that bruxing events can still be detected when the baseline biting force decreases.

In one or more embodiments described herein, the apparatus 100 may optionally configured to detect snoring. In such cases, the apparatus 100 may include one or more microphones configured to detect sound while the user is sleeping. The processing module 200 may be configured to determine whether the sound is snoring sound or not. If the processing module 200 determines that the detected sound is snoring sound, the processing module 200 may then generate a control signal to operate the feedback signal generator 240 to cause the feedback signal generator 240 to provide feedback to the user. In response to the feedback, the user may stop snoring.

In some cases, only a single microphone may be utilized for detecting snoring sound. Such microphone may be located at the connector 120 (e.g., coupled to an exterior surface of the connector 120), or at the processing module 200. In either case, the microphone is close to the mouth of the user, and can detect snoring sound effectively. The processing module 200 may compare the volume of the sound detected by the microphone against a volume threshold. If the volume of the detected sound exceeds the volume threshold, the processing module 200 may then determine that the detected sound is a snoring sound. Since snoring sound is generally louder than breathing noise, this technique can provide a cost effective and reliable way to detect snoring.

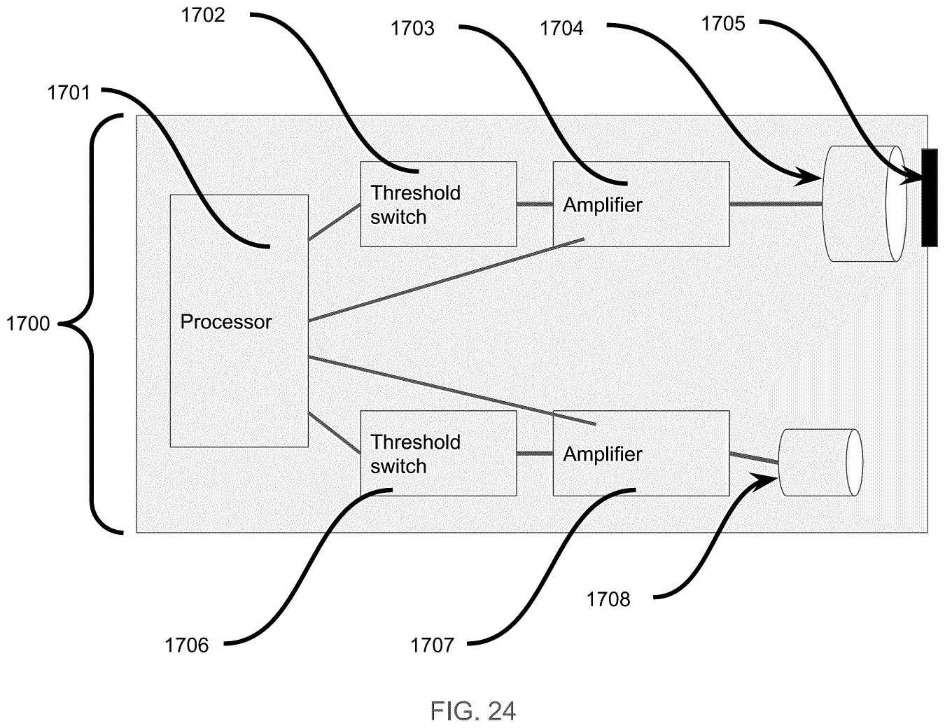

Alternatively, multiple microphones may be utilized to detect snoring. FIG. 24 illustrates a component 1700 having first microphone 1704 and second microphone 1708, wherein the component is configured to differentiate breathing sound from snoring sound. The component 1700 may be implemented as a part of the apparatus 100 in some embodiments. For example, the component 1700 may be a part of a processing unit (e.g., circuit) described herein. The first microphone 1704 is outside the processing module 200 and is located behind a water-proof mesh or diaphragm 1705. The first microphone 1704 may be coupled to an exterior of the processing module 200 or to the connector 120. The first microphone 1704 is configured to detect breathing noise (e.g., during exhale phase) from the user. The microphone signal from the first microphone 1704 will be high during exhales, because the user's breathing will stimulate this microphone that is in the path of the air flow. The second microphone 1708 is located in the processing module 200, and does not stimulate as readily with the breath air flow. However, the second microphone 1708 can pick up snoring sound. In some cases, the first microphone 1704 may be considered an external microphone, and the second microphone 1708 may be considered an internal microphone. The second microphone 1708 outputs a high signal for both breathing and snoring. On the other hand, the first microphone 1704 outputs a high signal for breathing, but a relatively lower signal for snoring. This difference is due to the different locations for the first and second microphones 1704, 1708. Therefore if both the second microphone 1708 and the first microphone 1704 provide high signals, the processing module 200 may determine that the sound is a breathing sound. If the second microphone 1708 provides a high signal, and the first microphone 1704 provides a relatively low signal, the processing module 200 may determine that the sound is a snoring sound. As shown in the figure, the component 1700 includes a comparator 1701 configured to perform comparison based on microphone signals from the first and second microphones 1704, 1708. Based on a result of the comparison, the comparator 1701 then determines whether the detected sound is from a snoring event or a breathing event. Due to the different signal strengths from the two microphones 1704, 1708 for breathing versus snoring, the component 1700 can differentiate between snoring and breathing without significant signal processing.

As shown in the figure, the component 1700 also includes a first amplifier 1703 for amplifying signals from the first microphone 1704, and a second amplifier 1707 for amplifying signals from the second microphone 1708. In other cases, the amplifiers 1703, 1707 may not be required.

The component 1700 also includes first threshold switch 1702 and second threshold switch 1706. The first threshold switch 1702 may be implemented as a first filter configured to filter out signals from the first microphone 1704 that are below a first filtering threshold. The second threshold switch 1706 may be implemented as a second filter configured to filter out signals from the second microphone 1708 that are below the first filtering threshold or a second filtering threshold that is different from the first filtering threshold. The first and second threshold switches 1702, 1706 are advantageous because they allow the processing module 200 to remain in a low-power state until breathing or snoring is detected. Thus, the component 1700 is a low-cost, low-power detector that is configured to pass the signals from amplifiers 1703, 1707 or from microphones 1704, 1708, through threshold switches, 1702, 1706. In other embodiments, the component 1700 may not include the threshold switches 1702, 1706.

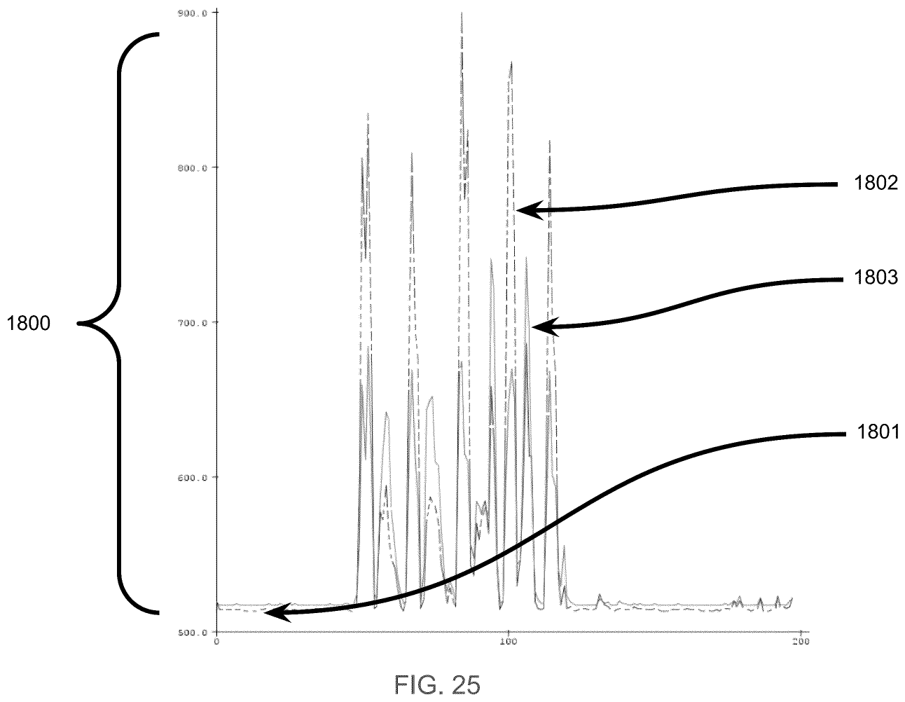

FIG. 25 illustrates microphone outputs 1800 from the component 1700 of FIG. 24. The plot 1802 represents voltage signal from the second microphone 1708. The plot 1803 represents voltage signal from the first microphone 1704. The x-axis of the plots 1802, 1803 indicates time, and the y-axis indicates power of microphone output voltage (which corresponds with sound volume/power of sound signal). The baseline noise level 1801 detected by the microphones when there is no snoring and no respiration is shown. During a snoring event, the plot 1802 indicates that the sound volume is high relative to the corresponding sound volume (i.e., at the same time point) in the plot 1803. During a breathing event, the plot 1803 indicates that the sound volume is high relative to the corresponding sound volume (i.e., at the same time point) in plot 1802. This shows that the due to microphone placement being so close to the mouth, it is easy and effective to distinguish respiration/snoring from the baseline. Because the microphone placement is so close to the mouth, the device may be more accurate at detecting snoring/breathing than a device further away (e.g., a mobile app running on a phone on the bedside).

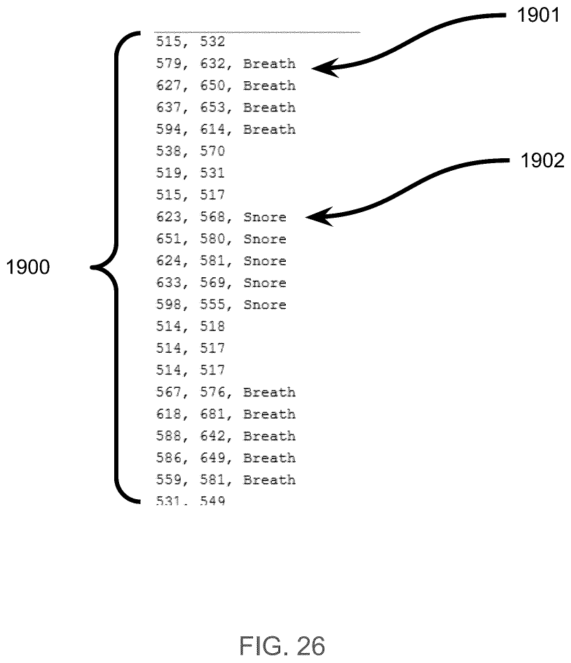

FIG. 26 illustrates data 1900 generated by the component 1700 of FIG. 24, particularly showing the component being capable of differentiating breathing sound from snoring sound. The left column of data is from the second microphone 1708 inside the processing module 200, and the right column of data is from the first microphone 1704 outside the processing module 200. As can be seen from the figure, when the first microphone signal is larger than the second microphone signal by a first threshold, the processing module 200 may determine that the event associated with the microphone outputs is a breathing event 1901. On the other hand, when the second microphone signal is larger than the first microphone signal by the first threshold (or a second threshold different from the first threshold), the processing module 200 may determine that the event associated with the microphone outputs is a snoring event 1902.

FIG. 27 illustrates another example of the processing module 200 of FIG. 1. The processing module 200 of FIG. 27 is the same as that of FIG. 1, except that the processing module 200 of FIG. 27 has a crescent shape. The crescent (moon) shape signifies sleep and indicates that the processing module 200 is for use during sleep. The crescent shape also prevents the processing module 200 from easily swallowed. The processing module 200 of FIG. 27 includes a post 2301 with the port 201 for coupling with the second end 124 of the tube (connector) 120, and the electrical port 250 for coupling with a charging/communication cable. As shown in the figure, the processing module 200 includes two protrusions 2303 (implementing a locking mechanism) configured to lock the tube (connector) 120 against the processing module 200 when the tube 120 is coupled with the port 201. The protrusions 2303 are located on opposite sides of the post 2301. The protrusions 2303 may be integrally formed with the post 2301, or may be secured to the post 2301 via an adhesive. The processing module 200 also includes an O-ring 2302 around the longitudinal axis of the port 201. The O-ring 2302 is configured to provide a seal.

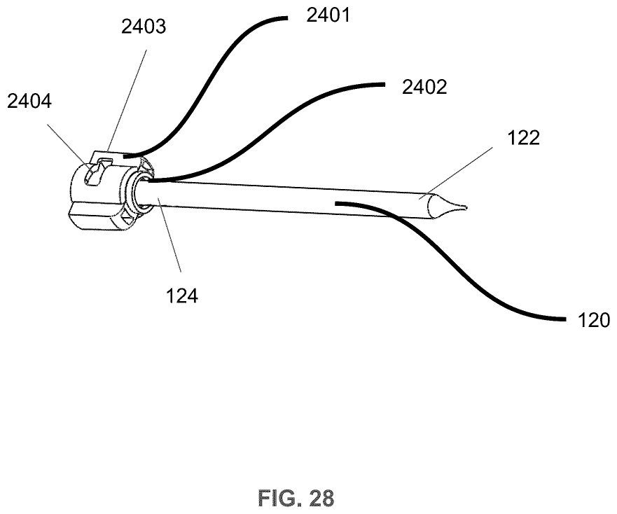

FIG. 28 illustrates an example of the tube (connector) 120 of FIG. 1. The tube (connector) 120 of FIG. 28 includes the first end 122 for coupling with the mouthpiece 300, and the second end 124 for removably coupling with the processing module 200 of FIG. 27. As shown in FIG. 28, the second end 124 of the tube 120 includes a locking feature 2401 configured to mate with the protrusions 2303 at the processing module 200. In particular, the locking feature 2401 includes a connector housing 2403 sized and shaped to receive the protrusions 2303 when the post 2301 at the processing module 200 is inserted into the tube 120 at the second end 124 of the tube 120. When the post 2301 is inserted into the tube 120, the O-ring 2302 provides a seal between the tube 120 and the post 2301. As shown in FIG. 28, the locking feature 2401 also includes opposing slots 2404 (one is shown in the figure, and the other one is on the opposite side of the locking feature 2401). The opposing slots 2404 are configured to receive the respective protrusions 2303 after the protrusions 2303 are inserted into the connector housing 2403, and after the connector housing 2403 is rotated around the longitudinal axis of the tube 120 relative to the processing module 200. The locking feature 2401 may be made from any of a variety of materials, such as plastic, polymer, alloy, etc. In one implementation, the locking feature 2401 may be made from an injection molded plastic. The locking feature 2401 may be secured to the second end 124 of the tube 120 by an adhesive 2402 (e.g., a biocompatible adhesive). Alternatively, the locking feature 2401 and the tube 120 may be integrally formed together.

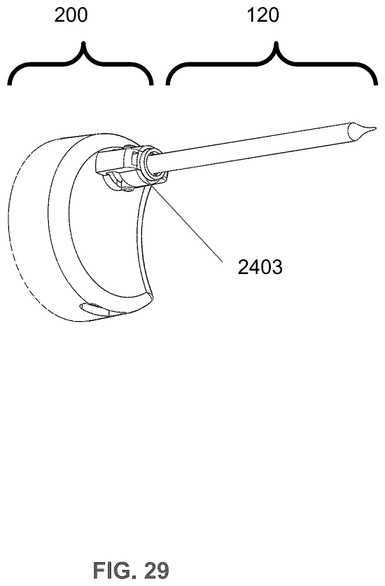

FIG. 29 illustrates the tube 120 of FIG. 28 being coupled with the processing module 200 of FIG. 27. During use, the second end 124 of the tube 120 is pressed onto the processing module 200 so that the post 2301 is inserted into the tube 120. At the same time, the protrusions 2303 of the locking mechanism are inserted into the connector housing 2403 at the second end 124 of the tube 120. The tube 120 is then twisted around the longitudinal axis of the tube 120 90-degree relative to the processing module 200 to lock the tube 120 against the processing module 200. The O-ring at the post 2301 ensure that there is an air-tight seal so that the pressure inside the tube 120 can transmitted to the pressure sensor inside the processing module 200 without leakage. The locking mechanism 2303 and the locking feature 2401 are advantageous because they establish a positive locking between the processing module 200 and tube (connector) 120.

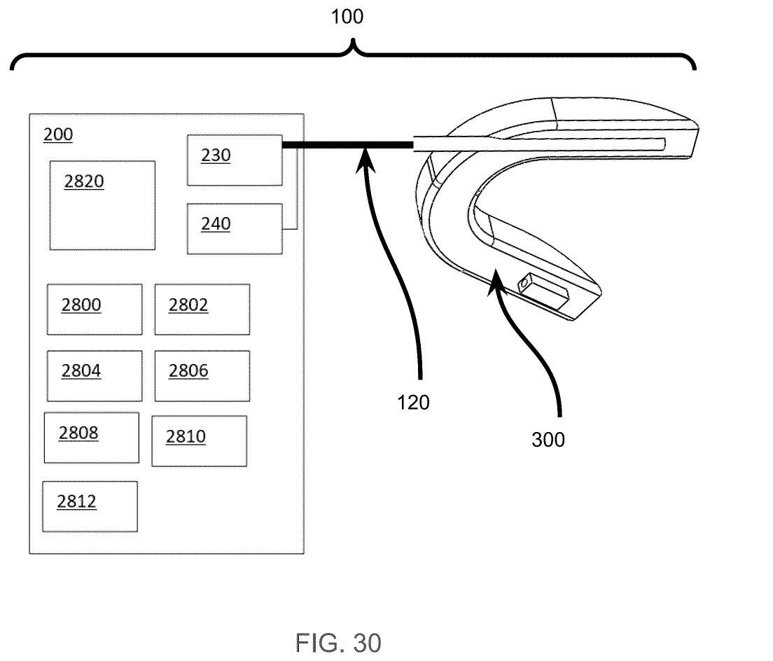

As described, the apparatus 100 of FIG. 1 may optionally include an accelerometer, a temperature sensor, a pulse oximeter, a force sensor, a speaker, a communication device for communication with the electronic device, and a non-transitory medium. FIG. 30 illustrates a variation of the apparatus 100 of FIG. 1, particularly showing the apparatus 100 having the above components. In particular, the apparatus 100 includes the mouthpiece 300, the tube (connector) 120, and the processing module 200, wherein the processing module 200 comprises the pressure sensor 230 and the feedback signal generator 240, like those described with reference to FIG. 1. The apparatus 100 of FIG. 30 further includes an accelerometer 2800, a temperature sensor 2802, a pulse oximeter 2804, a force sensor 2806, a speaker 2808, a communication device 2810 for communication with the electronic device 500, and a non-transitory medium 2812. The processing module 200 also includes a processing unit 2820 configured to communicate with the pressure sensor 230, the feedback signal generator 240, and components 2800-2812. In some cases, one or more of the components 2800-2812 may be implanted as part(s) of the processing unit 2820. Also, in some embodiments, the processing module 200 may not include all of the components 2800-2812. For example, one or more or all of the components 2800-2812 may be omitted in some embodiments. Furthermore, in some cases, the pressure sensor 230 and/or the feedback signal generator 240 may be implemented as part(s) of the processing unit 2820.

In the above embodiments, the apparatus 100 has been described as having the mouthpiece 300, the tube 120, and the processing module 200. However, it should be noted that the apparatus 100 may not include all of the above components. For example, in other embodiments, the apparatus 100 may be just the mouthpiece 300. In other embodiments, the apparatus 100 may be just the tube (connector) 120. In further embodiments, the apparatus 100 may include only the mouthpiece 300 and the tube 120. In still further embodiments, the apparatus 100 may be just the processing module 200. In further embodiments, the apparatus may include only the processing module 200 and the tube 120.

In some embodiments, the mouthpiece 300 may be provided and/or sold separately from the processing module 200 and/or from the connector (tube) 120. In some embodiments, the mouthpiece 300 includes: a body comprising one or more portions defining a space for accommodating one or more teeth of a user; and a first cavity 305 at least partly in the body, wherein a volume inside the first cavity 305 is variable in response to a compression applied to the body.

The detachability of the mouthpiece 300 from the tube (connector) 120 and/or from the processing module 200 is advantageous because it allows easy cleaning of the mouthpiece 300 and/or the processing module 200, allows replacement of the mouthpiece 300 when it is worn out, damaged, or lost, and/or allows replacement of the mouthpiece 300 when updated mouthpiece 300 with more advance features become available.

In some embodiments, the connector 120 may be provided and/or sold separately from the processing module 200 and/or from the mouthpiece 300. Such connector 120 is configured for coupling the mouthpiece 300 to the processing module 200. In some embodiments, such connector 120 includes: a tube having a first end, a second end opposite from the first end, and a tubular body extending between the first end and the second end; wherein the first end of the tube is configured to couple to the mouthpiece 300; wherein the second end of the tube is configured to couple to the processing module 200; and wherein the tube has a stiffness sufficient to transmit a vibrational force from the processing module 200 to the mouthpiece 300.

The detachability of the tube (connector) 120 from the mouthpiece 300 and/or from the processing module 200 is advantageous because it allows easy cleaning of the mouthpiece 300, the tube (connector) 120, and/or the processing module 200, and/or allows replacement of the tube 120 when it is worn out, damaged, or lost. In some embodiments, different tubes (connectors) 120 with different respective tube geometries or materials may be provided for the user's selection in order to optimize sensing. Also, the detachability of the tube (connector) 120 also allows the tube 120 to be replaced when new tube 120 with advanced features become available.

In some embodiments, the processing module 200 may be provided and/or sold separately from the mouthpiece 300 and/or from the connector (tube) 120. Also, the processing module 200 itself may be considered an apparatus. Accordingly, in some embodiments, an apparatus includes: a housing 210; and a pressure sensor 230 contained in the housing 210, the pressure sensor 230 configured to detect a pressure due to a biting force applied to a mouthpiece 300 by a user.

The detachability of the processing module 200 from the tube (connector) 120 and/or from the mouthpiece 300 is advantageous because it allows easy cleaning of the processing module 200, allows replacement of the processing module 120 when it is worn out, damaged, or lost, and/or allows replacement of the processing module 120 when updated processing module 120 with more advance features become available.

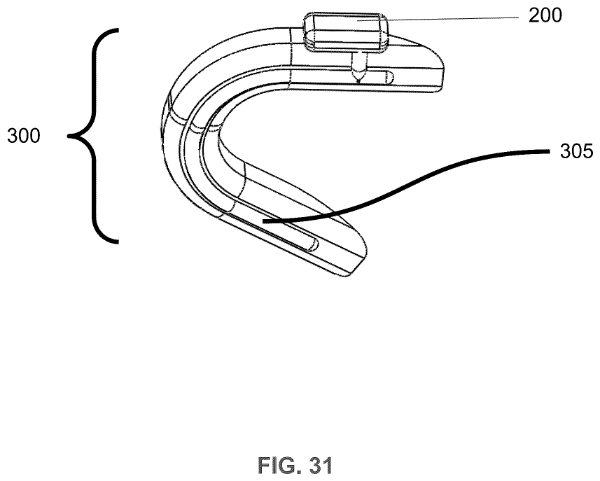

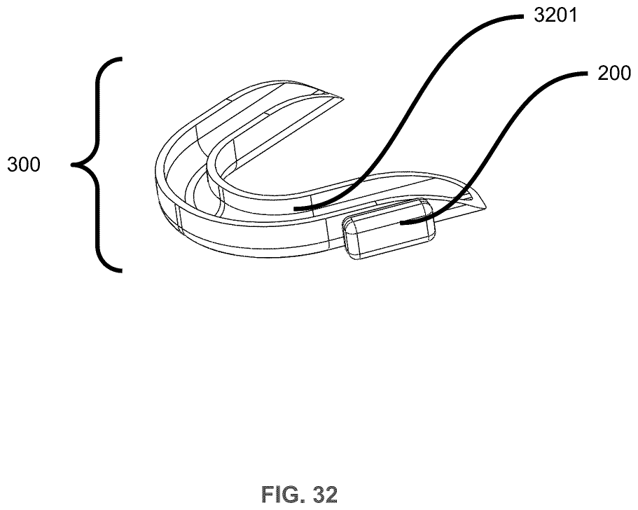

FIGS. 31-39 illustrates another variation of the apparatus 100 of FIG. 1. The apparatus 100 of FIG. 31 is the same as that of FIG. 1, except that the processing module 200 is integrated into the mouthpiece 300. FIGS. 31-32 show the mouthpiece 300 having the cavity 305 at the bite portion of the mouthpiece, and the processing module 200 attached to a side of the mouthpiece 300. The mouthpiece 300 includes a space 3201 for accommodating the teeth of the user. In other embodiments, instead of securing to a side (e.g., surface) of the mouthpiece 300, the processing module 200 may be embedded within the mouthpiece 300. In some cases, the processing module 200 may be sandwiched between two layers of the mouthpiece 300, or may be integrally molded with another part of the mouthpiece 300. Also, in some cases, the mouthpiece 300 itself may be considered as the housing of the processing module 200.

As shown in FIG. 31, the cavity 305 extends from a left side to the center and to the right side of the mouthpiece 300. Such configuration allows biting to be detected at different locations of the mouthpiece 300. In other cases, the cavity 305 may be shorter, and may be located at certain part of the mouthpiece 300, such as on the right side, on the left side, etc.

In the illustrated example, the cavity 305 of the mouthpiece 300 directly interfaces with the pressure sensor of the processing module 200. When the processing module 200 is coupled with the mouthpiece 300, it becomes a part of the mouthpiece 300, and the cavity 305 is in fluid communication with the pressure sensor in the processing module 200. In other cases, the cavity 305 may be communicatively coupled with the pressure sensor via one or more channels in the mouthpiece 300. In such cases, the one or more channels may be implemented using the tube 120 (with one or more lumens), which may be embedded inside the mouthpiece 300, or may be coupled to an exterior surface of the mouthpiece 300.

The cavity 305 of the mouthpiece 300 may be implemented using a tube that is secured to the surface of the mouthpiece 300, or that is embedded within the body of the mouthpiece 300. Thus, in some cases, the tube implementing the cavity 305 may be considered to be a part of the mouthpiece 300. In other cases in which the cavity 305 does not have a long elongated configuration, the cavity 305 may be implemented as a space defined between two layers of the mouthpiece 300. In further cases, the cavity 305 may also be implemented as a pocket in the mouthpiece 300.

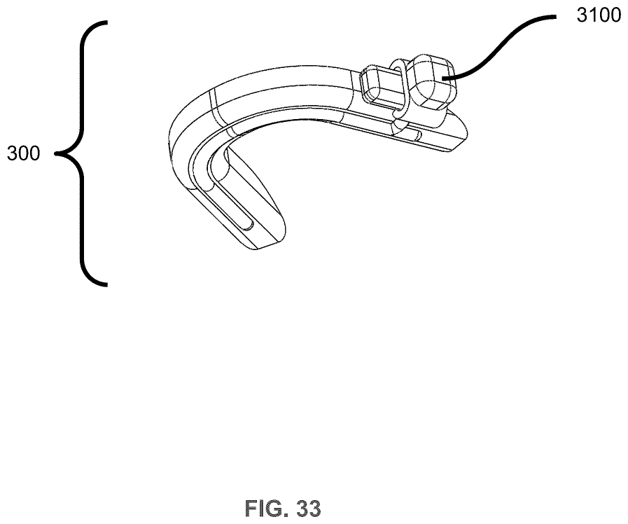

FIG. 33 shows the apparatus 100 of FIG. 31, further having a charger 3100 removably coupled to the processing module 200. The charger 3100 is configured to provide charging power wirelessly to the processing module 200. In other embodiments, the charger 3100 is not needed, and the processing module 200 may be charged using the electrical port 250 described herein.

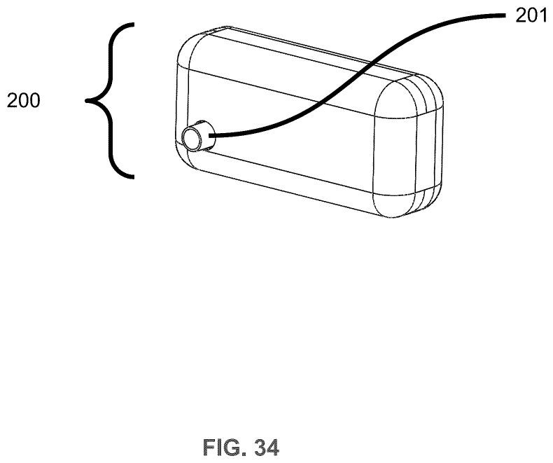

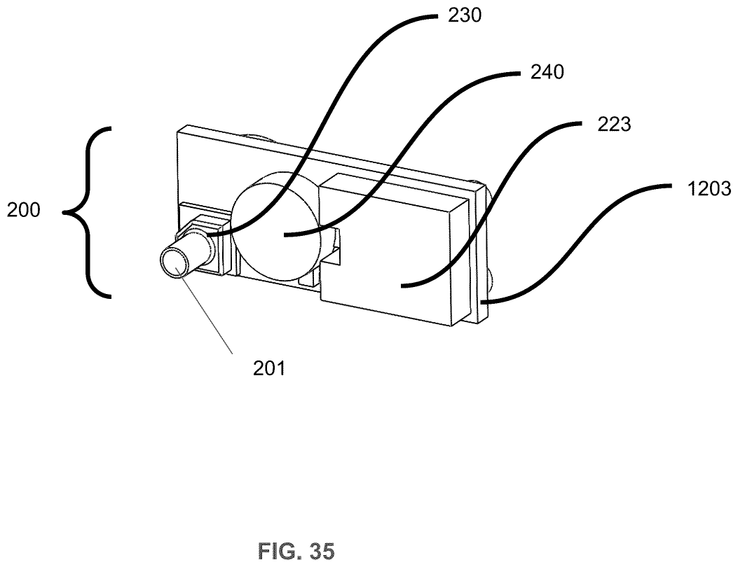

FIG. 34 shows an example of the processing module 200 of FIG. 31. The processing module 200 is miniature in size, and includes the port 201 for coupling with the cavity 305 of the mouthpiece 300 when the processing module 200 is attached to the mouthpiece 300. FIG. 35 shows the components inside the processing module 200 of FIG. 34. The processing module 200 includes the battery 223, the feedback signal generator 240 (e.g., motor), the pressure sensor 230, and the PCB 1203. The functions of these components are described previously.

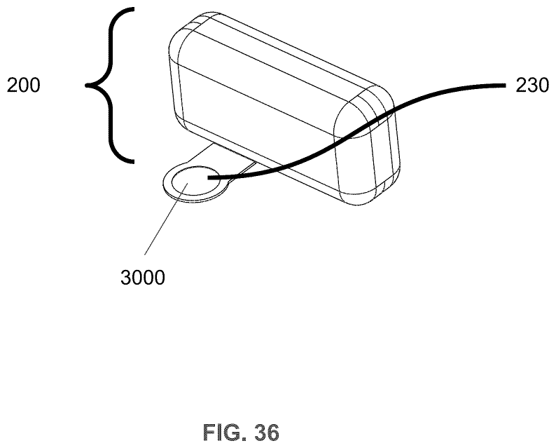

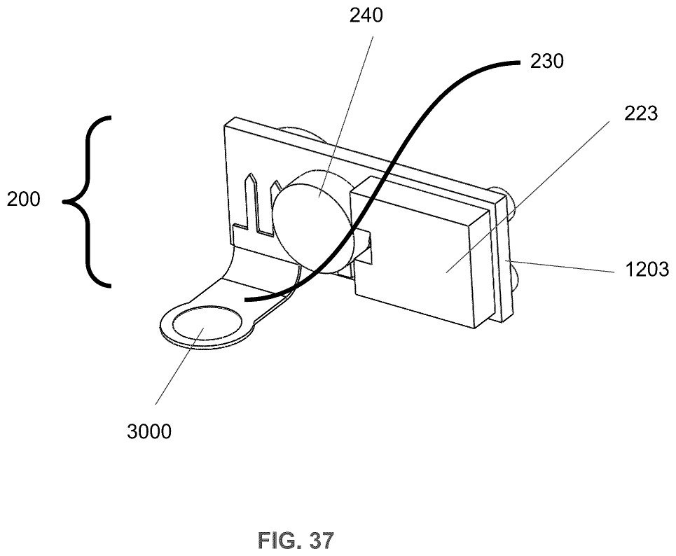

In other embodiments, instead of the pressure sensor, sensor 230 may be a force sensor. FIG. 36 shows another example of the processing module 200 of FIG. 31. The processing module 200 is miniature in size, and includes a force sensor 230 for coupling with the mouthpiece 300 when the processing module 200 is attached to the mouthpiece 300. FIG. 37 shows the components inside the processing module 200 of FIG. 36. The processing module 200 includes the battery 223, the feedback signal generator 240 (e.g., motor), the force sensor 230, and the PCB 1203. The functions of these components are described previously. The force sensor 230 includes a sensing region 3000 configured to be inserted into a cavity (e.g., slot) of the mouthpiece 300. The sensing region 3000 is configured to be placed behind a bite portion of the mouthpiece 300 so that when the user applies biting force, the sensing region 3000 will be compressed between an upper tooth and a lower tooth. In other cases, the sensing region 3000 may have a different shape from that shown, and may detect biting force between multiple upper teeth and multiple lower teeth.

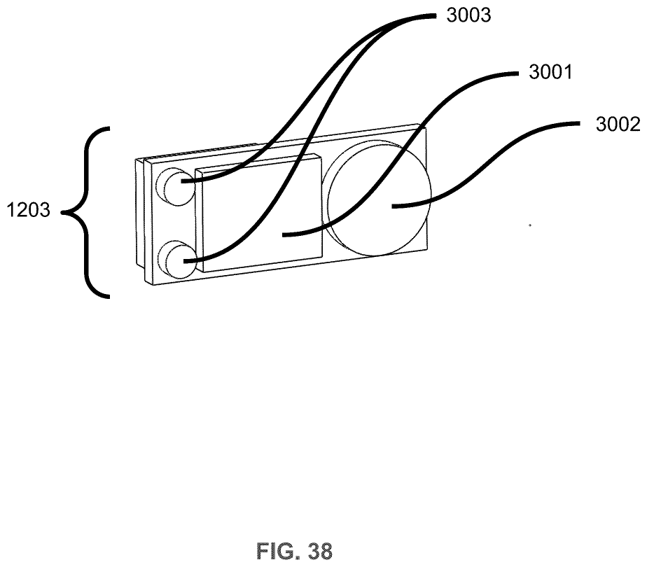

FIG. 38 shows the back side of the PCB 1203. The PCB 1203 includes the processor and Bluetooth radio 3001. The PCB 1203 also includes a coil 3002 used to achieve wireless charging. Since the processing module 200 is configured for placement in the mouth of the user, a non-contact charging is utilized. The PCB 1203 also includes magnetic elements 3003 configured to align the coil 3002 with a corresponding charging element during charging. In other cases, the PCB 1203 may include only one magnetic element 3003, or more than two magnetic elements 3003. Also, in other cases, the magnetic element(s) 3003 may be implanted away from the PCB 1203, and may be located at other part(s) of the processing module 200.

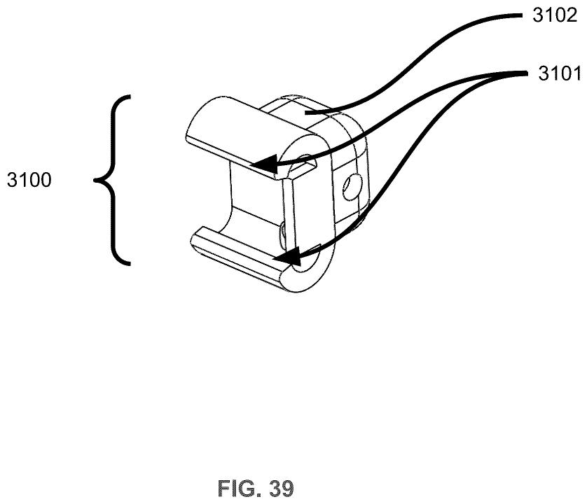

FIG. 39 shows the charger 3100 for the apparatus 100 of FIG. 31. The charger 3100 is configured to clip onto the processing module 200 via retaining features 3101 (e.g., clips). The charger 3100 includes a PCBA in the charger housing 3102. During use, wireless power is transmitted via the PCBA to the processing module 200.

The apparatus 100 of FIG. 31 may include other features described herein. For example, the apparatus 100 may optionally include an accelerometer, a temperature sensor, a pulse oximeter, a force sensor, a speaker, a communication device for communication with the electronic device 500, a non-transitory medium, etc., or any combination of two or more of the foregoing.

In the above embodiments, the processing module 200 is described as having a housing. In other embodiments, the processing module 200 may not include any housing. For example, in other embodiments, the processing module 200 may include a processing unit (e.g., processor, PCB, sensor(s), feedback signal generator, etc.) that is contained within the mouthpiece 300. In further embodiments, if the processing unit is implemented at the mouthpiece 300, the processing unit may include a housing.

Also, in the above embodiments, the processing module 200 and the electronic device 500 are described as having certain features. In other embodiments, one or more of the features described with reference to the electronic device 500 may be implemented at the processing module 200. Also, in other embodiments, one or more of the features described with reference to the processing module 200 may be implemented at the electronic device 500.

In addition, in the above embodiments, the apparatus 100 is described as being configured to detect a characteristic due to biting force applied by the user. In other embodiments, the apparatus 100 may not have the bite-detection characteristic. For example, in other embodiments, the apparatus 100 may be an apnea treatment device configured to address apnea condition of the user. In such cases, the mouthpiece 300 may not include the cavity 305 for detecting bite pressure. The processing module 200 is configured to detect an apnea event while the user is wearing the mouthpiece 300, and is configured to operate the feedback signal generator 240 in response to a detected apnea event. The feedback signal generator 240 may provide vibration, light, sound, electric shock, air-puff, etc., or any of two or more of the foregoing. The processing module 200 may be configured to detect the apnea event based on an output from a microphone and/or an output from a pulse oximeter. For example, if the output from the microphone indicates that a detected sound is an apnea sound, the apparatus 100 may then provide the feedback. As another example, if the output from the microphone indicates that the user has not breathed for a certain duration threshold, and/or if the output from the pulse oximeter is below a certain level, then the apparatus 100 may provide the feedback.

Although particular features have been shown and described, it will be understood that they are not intended to limit the claimed invention, and it will be made obvious to those skilled in the art that various changes and modifications may be made without departing from the scope of the claimed invention. The specification and drawings are, accordingly to be regarded in an illustrative rather than restrictive sense. The claimed invention is intended to cover all alternatives, modifications and equivalents.

What is claimed:

1. A mouthpiece comprising:
a body comprising one or more portions defining a space for accommodating one or more teeth of a user; and
a first cavity at least partly in the body, wherein a volume inside the first cavity is variable in response to a compression applied to the body.

2. The mouthpiece of claim 1, wherein the one or more teeth comprise one or more upper teeth, and wherein the space is configured to accommodate the one or more upper teeth; or
wherein the one or more teeth comprise one or more lower teeth, and wherein the space is configured to accommodate the one or more lower teeth.

3. The mouthpiece of claim 1, further comprising a second cavity, wherein the first cavity is at one of a left side or a right side of the body, and the second cavity is at another one of the left side or the right side of the body.

4. The mouthpiece of claim 3, further comprising a spacer configured for placement in the first cavity or the second cavity.

5. The mouthpiece of claim 1, wherein the mouthpiece is a customized mouthpiece.

6. The mouthpiece of claim 1, further comprising a first tube coupled to, and/or extending from, the body.

7. The mouthpiece of claim 6, wherein the first tube is removably coupled to the body.

8. The mouthpiece of claim 6, wherein the first tube is integrally formed with the body.

9. The mouthpiece of claim 6, wherein the first tube is on one of a left side or a right side of the body.

10. The mouthpiece of claim 9, further comprising a second tube, wherein the second tube is on another one of the left side or the right side of the body.

11. The mouthpiece of claim 1, wherein the body comprises at least two layers of materials laminated together.

12. The mouthpiece of claim 1, further comprising a processing module.

13. The mouthpiece of claim 1, further comprising a sensor and a feedback signal generator, wherein the cavity is coupled to the sensor.

14. The mouthpiece of claim 1, further comprising a pulse oximeter.

15. The mouthpiece of claim 1, further comprising a temperature sensor.

16. The mouthpiece of claim 1, further comprising an accelerometer.

17. The mouthpiece of claim 1, further comprising a communication device.

18. The mouthpiece of claim 1, further comprising a non-transitory medium storing data associated with an operation of the mouthpiece.

19. An apparatus comprising the mouthpiece of claim 1 and a connector.

20. The apparatus of claim 19, wherein the connector comprises a tube;
wherein the tube has a first end, a second end opposite from the first end, and a tubular body extending between the first end and the second end;
wherein the first end of the tube is configured to couple to the mouthpiece; and
wherein the second end of the tube is configured to couple to a processing module.

21. The apparatus of claim 20, wherein the tube has a stiffness sufficient to transmit a vibrational force from the processing module to the mouthpiece.

22. The apparatus of claim 20, further comprising the processing module, wherein the processing module comprises a sensor configured to detect a characteristic due to a bruxing event, a snoring event, or an apnea event.

23. The apparatus of claim 22, wherein the processing module comprises a feedback signal generator configured to provide a feedback based on the detected characteristic.

24. A connector for coupling a mouthpiece to a processing module comprising:
a tube having a first end, a second end opposite from the first end, and a tubular body extending between the first end and the second end;
wherein the first end of the tube is configured to couple to the mouthpiece;
wherein the second end of the tube is configured to couple to the processing module; and
wherein the tube has a stiffness sufficient to transmit a vibrational force from the processing module to the mouthpiece.

25. The connector of claim 24, wherein the first end of the tube is configured to detachably couple to the mouthpiece.

26. The connector of claim 24, wherein the second end of the tube is configured to detachably couple to the processing module.
